(19) United States
(12) Patent Application Publication
(10) Pub. No.: US 2026/0098057 A1
(43) Pub. Date: Apr. 9, 2026

(54) CRYSTALLISATION OF A PROTEIN CAPABLE OF CRYSTALLIZING UNDER SALTING-IN CONDITIONS USING MULTIPLE PROTEIN FEEDS

(71) Applicant: ARLA FOODS AMBA, Viby J (DK)

(72) Inventors: Hans BERTELSEN, Viby J (DK); Kasper BØGELUND LAURIDSEN, Viby J (DK); Nikolaj DRACHMANN, Viby J (DK)

(21) Appl. No.: 19/099,236

(22) PCT Filed: Aug. 10, 2022
(86) PCT No.: PCT/EP2022/072459

§ 371 Date: Jan. 28, 2025

(51) Int. Cl. C07K 1/30; C07K 14/47; C07K 14/555; C07K 14/605; C07K 14/62; C07K 16/11; C07K 16/18; C07K 16/24; C07K 16/28; C07K 16/32; C12N 9/02; C12N 9/04; C12N 9/08; C12N 9/16; C12N 9/20; C12N 9/24; C12N 9/26; C12N 9/34; C12N 9/36; C12N 9/38; C12N 9/42; C12N 9/44; C12N 9/50; C12N 9/88; C12N 9/90

(52) U.S. Cl. CPC C07K 1/306; C07K 14/4717; C07K 14/555; C07K 14/605; C07K 14/62; C07K 16/11; C07K 16/18; C07K 16/241; C07K 16/2818; C07K 16/2827; C07K 16/2863; C07K 16/2866; C07K 16/2875; C07K 16/2887; C07K 16/32; C12N 9/0006; C12N 9/0061; C12N 9/0065; C12N 9/16; C12N 9/20; C12N 9/2402; C12N 9/2414; C12N 9/2428; C12N 9/2437; C12N 9/2457; C12N 9/2462; C12N 9/2471; C12N 9/50; C12N 9/88; C12N 9/90; C12Y 101/03004; C12Y 110/03002; C12Y 111/01014; C12Y 301/01003; C12Y 302/01001; C12Y 302/01003; C12Y 302/01004; C12Y 302/01008; C12Y 302/01011; C12Y 302/01017; C12Y 302/0102; C12Y 302/01023; C12Y 302/01041; C12Y 302/01067; C12Y 302/01108; C12Y 304/21001; C12Y 304/23004

(57) ABSTRACT

The present invention relates to an improved method of producing products comprising a target protein (TP) capable of crystallising under salting-in conditions by crystallisation of the TP. The invention makes use of at least two different TP-containing protein feeds which are mixed to prepare a supersaturated protein solution in which TP is crystallized. One of the protein feeds must have a pH of at least the pH providing the maximum crystallisation yield of the TP (pH_{MCY,TP}) plus 0.1 and one of the protein feeds must have a pH of at most pH_{MCY,TP} minus 0.1.

### FIELD OF THE INVENTION

The present invention relates to an improved method of purifying a protein (the target protein, abbreviated TP) that is capable of crystallising under salting-in conditions. Crystallisation under salting-in conditions are normally only possible within a certain pH-range. The invention makes use of at least two different TP-containing protein feeds, which have pH-values that ensure that they are not supersaturated with respect to the TP. The TP-containing protein feeds are then mixed to provide a mixture which has a pH where crystallisation under salting-in conditions is feasible and which is supersaturated with respect to the TP. The invention requires the use of i) a protein feed which has a pH above the pH providing the maximum crystallisation yield of the TP (pH_{MCY,TP}), referred to as a “type A feed”, and ii) a protein feed having a pH below the pH_{MCY,TP}, referred to as a “type B feed”.

### BACKGROUND OF THE INVENTION

The concept of protein fractionation is well-known in the art and has been developed during the last decades to an array of technologies for preparing products enriched with various protein species each having specific properties and characteristics.

Isolation of beta-lactoglobulin (BLG) from milk serum or whey is the subject of a number of publications and typically involves multiple separation steps and often chromatographic techniques to arrive at a purified beta-lactoglobulin product.

WO2018/115520 describes industrial scale separation of BLG from protein solutions by crystallisation in salting-in mode and in the pH range 5-6. However, WO2018/115520 does not disclose preparing a supersaturated whey protein solution by mixing BLG-containing type A protein feed(s) and type B protein feed(s) as defined by the present invention.

WO 2020/002422 describes production of alpha-lactalbumin enriched whey protein products by industrial scale removal of BLG from whey protein solutions using BLG crystallisation in salting-in mode and at a pH in the range of 5-6. However, WO 2020/002422 does not disclose preparing a supersaturated whey protein solution by mixing BLG-containing type A protein feed(s) and type B protein feed(s) as defined by the present invention.

de Jongh et al (Mild Isolation Procedure Discloses New Protein Structural Properties of β-Lactoglobulin, J Dairy Sci., vol. 84 (3), 2001, pages 562-571) described purification of BLG from freshly milked milk by low temperature acid coagulation of casein and by subjecting the obtained acid whey to a combination of affinity chromatography (DEAE Sepharose) and gel permeation chromatography. The obtained BLG product was stated to contain 0.985 g beta-lactoglobulin per 1 g protein.

Slack et al (Journal of Food Processing and Preservation, vol. 10, 1986, pages 19-30) explored a different approach and prepared BLG-enriched precipitates by pH adjusting demineralised acid whey and sweet whey to pH 4.65 and separating the formed precipitate by centrifugation and decantation. The obtained precipitate pellets were described as being relatively insoluble and contained a significant amount of protein impurities in additional BLG. No crystal formation was observed. It should be noted that the BLG precipitates that may form at pH 4.65 are not BLG crystals.

Palmer (Crystalline Globulin from Cow's Milk, J. Biol. Chem., Vol. 104, 1934, pages 359-372) reported a laborious and time consuming process for producing protein crystals based on acid whey using several sequences of salt precipitation of unwanted proteins, pH-adjustments and dialysis to remove other unwanted proteins. Finally, when a highly purified BLG solution had been obtained, BLG was crystallized. The process lasted more than 12 days and required addition of toluene. The procedures disclosed in Palmer are therefore incompatible with safe food production and provides products that are clearly not edible.

Aschaffenburg et al (Improved Method for the Preparation of Crystalline beta-Lactoglobulin and alpha-Lactalbumin from Cow's Milk, Bioch., vol. 65, 1957, pages 273-277) discloses an improved process relative to the process of Palmer's process, which improvement allows for preparation of beta-lactoglobulin crystals in the order of few days instead of weeks. However, the improved method still requires removal of unwanted proteins prior to crystallisation and furthermore employs toluene for the crystallisation, which makes it incompatible with safe food production.

JP H10 218755 A discloses production of cosmetic products containing a melanin-producing inhibitor, which comprises BLG as an active ingredient. The document furthermore suggests that BLG e.g. may be isolated by the following process: Hydrochloric acid is added to milk to precipitate casein followed by filtration to obtain whey. The pH of the whey is adjusted to 6.0 and ammonium sulfate is added in an amount of half saturation; the precipitated protein is removed by salting out, and a filtrate is recovered. The filtrate is saturated with ammonium sulfate and the precipitated protein is recovered. The recovered protein is again dissolved in water and dialyzed at pH 5.2 to separate the crystals, and β-lactoglobulin is prepared at a proportion of about 1.8 g from 1 L whey. However, the general process steps of the proposed process described in JP H10 218755 A are insufficient to lead to the formation of BLG crystals. The document therefore does not contain an enabling disclosure of crystallisation of BLG or of BLG crystals.

U.S. Pat. No. 2,790,790 discloses a process for precipitation of proteins from solution, and more particularly to the fractional precipitation of relatively unconjugated proteins from aqueous solution by the use of sodium chloride as the precipitant. The process is suggested to be useful for isolating BLG by NaCl-induced precipitation at pH 3.6-3.8. In example II of the document, it is suggested that the NaCl-precipitate may be dialysed in the usual manner to form crystalline beta-lactoglobulin. However, U.S. Pat. No. 2,790,790 does not demonstrate that formation of BLG crystals at pH 3.6-3.8 is actually possible and contains no reference to meaning of “the usual manner” of dialyzing a BLG precipitate. The document therefore does not contain an enabling disclosure of crystallisation of BLG or of BLG crystals.

### SUMMARY OF THE INVENTION

It has previously been discovered that beta-lactoglobulin (BLG) can be efficiently isolated in industrial scale by crystallisation (see PCT application WO2018/115520). This crystallisation method requires processing and handling of large quantities of supersaturated protein solution.

The present inventors subsequently discovered that large scale implementation of WO2018/115520 could lead to undesired crystallisation of BLG before the intended crystallisation of the process. This is e.g. a problem if the BLG crystallisation takes place in membrane filtration units or other equipment, which is sensitive to the presence of particulate matter.

The present inventors furthermore discovered that the isolation of BLG by crystallisation can be improved and made more robust by preparing the supersaturated protein solution by mixing two or more BLG-containing protein feeds which have no or only a low level of BLG supersaturation as such, but which provide a higher degree of supersaturation when combined. One of these protein feeds should have a pH of at least 5.6 and more preferably at least 6.0 and the other should have a pH of at most 5.4 and more preferably at most 5.0. When combined, the two feeds provide a supersaturated BLG protein solution having a pH close to 5.5, which the inventors have found to be the optimum pH for the yield of BLG crystallisation.

The step of combining the protein feeds is preferably performed by mixing the feeds and does not required use of equipment that is sensitive to crystallisation of protein. This invention therefore reduces or even completely avoids the risk of early and unwanted BLG crystallisation, which may lead to clogging and malfunction of the involved process equipment. This invention was described in PCT/EP2022/053010.

The present inventors have furthermore appreciated that the technique of protein crystallizing described in PCT/EP2022/053010 can be applied to other protein species capable of crystallizing under salting-in conditions, such as e.g. enzymes and immunoglobulins.

A schematic illustration of the formation of the supersaturated protein solution is shown in FIG. 1, where one or more type B protein feed(s) (having a pH less than pH_{MCY,TP} and preferably lower) are mixed with one or more type A protein feed(s) (having a pH higher than pH_{MCY,TP}) in amounts sufficient to provide the initial protein solution which has a pH in the range of pH_{MCY,TP}±0.7 and is supersaturated with respect to BLG.

The inventors have found this approach very advantageous for large scale crystallisation of BLG as the individual type A and B protein feeds can be produced with no or only very limited risk of early (and hence unwanted) BLG crystallisation. However, as soon as the type A protein feed(s) and type B feed(s) are mixed the supersaturated initial protein solution is formed.

FIG. 2 illustrates schematically how the yield of BLG crystallisation depends on the pH of the protein solution in which the crystallisation is to take place. The inventors have found the optimum pH to be approx. 5.5. Therefore, by mixing more acidic type B protein feed(s) with less acidic type A protein feed(s), an initial protein solution is obtained which is supersaturated with respect to BLG. If the type A protein feed(s) and/or the type B protein feed(s) are slightly supersaturated with respected to BLG, the initial protein solution must be even more supersaturated.

Guided by the disclosures of the present application and the common general knowledge, a skilled person can prepare type A protein feed(s) and type B protein feed(s) and mix them to provide initial protein solutions as described herein.

Thus, an aspect of the invention pertains to a method of preparing a product, preferably an edible or pharmaceutically acceptable product, comprising a target protein (TP) capable of crystallizing under salting-in conditions, the method comprising the steps of:
a) preparing an initial protein solution comprising the TP, said initial protein solution is supersaturated with respect to the TP and has a pH at which the TP is capable of crystalizing under salting-in conditions, preferably in the range of +/−0.7 pH-units of the pH that provides the maximum crystallisation yield of the TP (pH_{MCY,TP}), and most preferably in the range of +/−0.5 pH-units of the pH_{MCY,TP},
b) crystallising the TP in the supersaturated, initial protein solution in salting-in mode thereby obtaining a TP crystal-containing solution, and
c) preferably, separating TP crystals from the remaining liquid of the TP crystal-containing solution,
wherein step a) involves preparing the initial protein solution by combining one or more type A protein feed(s) with one or more type B protein feed(s),
and wherein:
a “type A protein feed” is defined as a protein feed which comprises the TP and has a pH that is at least 0.1 pH unit higher than the pH_{MCY,TP}, and
a “type B protein feed” is defined as a protein feed which comprises the TP and has a pH that is at least 0.1 pH unit lower than the pH_{MCY,TP},
and preferably with the proviso that the TP is not a beta-lactoglobulin.

The TP is preferably present in crystallised and/or isolated form in the product obtained by the method.

In the present context the terms “product”, “product comprising the TP” and “TP product” are used interchangeably and pertain to the product of the present method.

The “pH providing the maximum crystallisation yield of the TP”, abbreviated “pH_{MCY,TP}” is determined as follows:

Determination of the pH that Provides the Maximum Crystallization Yield of the TP:
i) Prepare a stock solution of the TP, the stock solution having the same composition as the intended initial protein solution of step a) of the method of the invention but a pH at the isoelectric point (pI) of the TP. The weight percentage of the TP (including free, reversibly precipitated and crystallised TP) in the stock solution is determined.
ii) Prepare a set of samples, each sample having a different pH so the set of samples span the pH range pH 2-11 in steps of 0.1 pH-unit. The set of samples is prepared by metering the same amount of the stock solution into glass beakers and adjusting their pH by addition of the smallest possible weight of 0.5 M HCl (aq) or 0.5 M NaOH (aq) under agitation to reach the desired pH in each sample.
iii) The temperature of the samples is adjusted to the minimum temperature that will be used during step b) of the method of the invention, however not less than 0 degrees C.
iv) Each sample is seeded with TP crystal material the added in an amount to make the TP crystal material constitute 1% w/w of total weight of the TP in the seeded sample.
v) The seeded samples are then stored for 24 hours maintaining the minimum temperature mentioned in step iii) under gentle agitation. Subsequently, the samples are centrifuged at 10000 g for 60 minutes at the temperature of step iii); the supernatant and the precipitate are recovered separately. The precipitate of each sample is inspected by microscopy using polarized light to check for the presence of TP crystal material (which will be birefringent). If birefringent material is found in the precipitate, the corresponding supernatant is analysed (preferably by HPLC) to determine its weight percentage of the TP. Subsequently, the weight of TP in the recovered supernatant and the weight of TP in the seeded sample are calculated.
vi) The crystallization yield of each TP-crystal-containing sample is calculated as follows:

$$\frac{( weight of TP in the seeded sample ) - ( weight of TP in the supernatant )}{( weight of TP in the seeded sample )} \times 100 = crystallization yield \%$$

vii) pH_{MCY,TP} is determined as the pH of the sample that provides the highest crystallisation yield.
In the context of the present invention the phrase “a pH in the range of +/−x pH-units of the pH_{MCY,TP}” means that the pH is in the range of (pH_{MCY,TP}−x) to (pH_{MCY,TP}+x). For example, if x=0.5 and pH_{MCY,TP}=5.7 the pH should be in the range (5.7−0.5) to (5.7+0.5), i.e. in the range of 5.2-6.2.

Another aspect of the invention pertains to the TP product obtainable by the present method.

### BRIEF SUMMARY OF THE FIGURES

FIG. 1 illustrates step a) of the method where type A protein feed(s) is mixed with type B protein feed(s) to prepare the initial protein solution.

FIG. 2 is a schematic illustration of how the pH of the type A protein feed(s), type B protein feed(s) and the initial protein solution are located relative to the optimum pH for isolation of BLG by crystallisation.

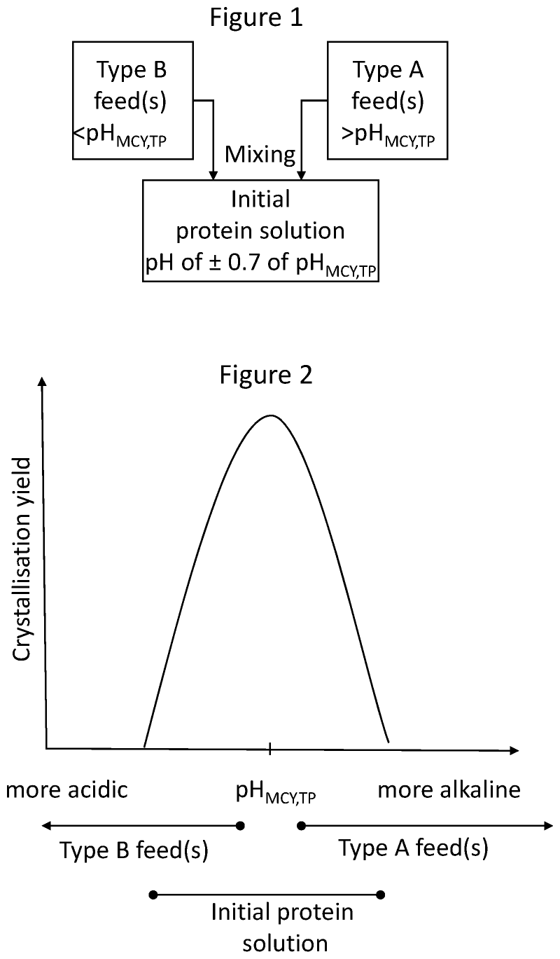

### DETAILED DESCRIPTION

As mentioned above, an aspect of the invention pertains to a method of preparing a product, preferably an edible or pharmaceutically acceptable product, comprising a target protein (TP) capable of crystallizing under salting-in conditions, the method comprising the steps of:
a) preparing an initial protein solution comprising the TP, said initial protein solution is supersaturated with respect to the TP and has a pH at which the TP is capable of crystalizing under salting-in conditions, preferably in the range of +/−0.7 pH-units of the pH that provides the maximum crystallisation yield of the TP (pH_{MCY,TP}), and most preferably in the range of +/−0.5 pH-units of the pH_{MCY,TP},
b) crystallising the TP in the supersaturated, initial protein solution in salting-in mode thereby obtaining a TP crystal-containing solution, and
c) preferably, separating TP crystals from the remaining liquid of the TP crystal-containing solution,
wherein step a) involves preparing the initial protein solution by combining one or more type A protein feed(s) with one or more type B protein feed(s),
and wherein:
a “type A protein feed” is defined as a protein feed which comprises the TP and has a pH that is at least 0.1 pH unit higher than the pH_{MCY,TP}, and
a “type B protein feed” is defined as a protein feed which comprises the TP and has a pH that is at least 0.1 pH unit lower than the pH_{MCY,TP},
and preferably with the proviso that the TP is not a beta-lactoglobulin.

In the context of the present invention, the term “edible product” pertains to a product that is safe for human consumption and use as a food ingredient and that does not contain problematic amounts of toxic components such as toluene or other unwanted organic solvents.

In the context of the present invention, the term “crystal” pertains to a solid material whose constituents (such as atoms, molecules or ions) are arranged in a highly ordered microscopic structure, forming a crystal lattice that extends in all directions. TP crystals are protein crystals that primarily contains TP arranged in a highly ordered microscopic structure, forming a crystal lattice that extends in all directions. The TP crystals may e.g. be monolithic or polycrystalline and may e.g. be intact crystals, fragments of crystals, or a combination thereof. Fragments of crystal are e.g. formed when intact crystals are subjected to mechanical shear during processing. Fragments of crystals also have the highly ordered microscopic structure of crystal but may lack the even surface and/or even edges or corners of an intact crystal. See e.g. FIG. 18 of WO2018115520 A1 for an example of many intact BLG crystals and FIG. 13 of WO2018115520 A1 for an example of fragments of BLG crystals. In both cases the BLG crystal or crystal fragments can be identified visually as well-defined, compact and coherent structures using light microscopy. TP crystal or crystal fragments are often at least partially transparent. Protein crystals are furthermore known to be birefringent and this optical property can be used to identify unknown particles as having crystal structure. Non-crystalline TP aggregates, on the other hand, appear as poorly defined, non-transparent, and as open or porous lumps of irregular size.

In the context of the present invention, the term “crystallise” pertains to formation of protein crystals. Crystallisation may e.g. happen spontaneously or be initiated by the addition of crystallisation seeds.

The product preferably comprises the TP in crystallised and/or isolated form. An edible product that comprises TP in isolated form comprises at least 50% w/w TP relative to total solids. An edible product that comprises TP in crystallised form comprises at least some TP crystals, and preferably a significant amount of TP crystals.

TP crystals can often be observed by microscopy and may even reach a size which makes them visible by eye.

In the context of the present invention, a liquid which is “supersaturated” or “supersaturated with respect to TP” contains a concentration of dissolved TP which is above the saturation point of TP in that liquid at the given physical and chemical conditions. The term “supersaturated” is well-known in the field of crystallisation (see e.g. Gerard Coquerela, “Crystallization of molecular systems from solution: phase diagrams, supersaturation and other basic concepts”, Chemical Society Reviews, p. 2286-2300, Issue 7, 2014) and supersaturation can be determined by a number of different measurement techniques (e.g. by spectroscopy or particle size analysis). In the context of the present invention, supersaturation with respect to TP is determined by the following procedure.

Procedure for Testing Whether a Liquid at a Specific Set of Conditions is Supersaturated with Respect to TP:
a) Transfer a 50 ml sample of the liquid to be tested to a centrifuge tube (VWR Catalogue no. 525-0402) having a height of 115 mm, an inside diameter of 25 mm and a capacity of 50 mL. Care should be taken to keep the sample and subsequent fractions thereof at the original physical and chemical conditions of the liquid during steps a)-h).
b) The sample is immediately centrifuged at 3000 g for 3.0 minutes with max. 30 seconds acceleration and max 30 seconds deceleration.
c) Immediately after the centrifugation, transfer as much as possible of the supernatant (without disturbing the pellet if a pellet has formed) to a second centrifuge tube (same type as in step a)
d) Take a 0.05 mL subsample of the supernatant (subsample A)
e) Add 10 mg TP crystals (at least 98% pure TP relative to total solids) having a particle size of at most 200 micron to the second centrifuge tube and agitate the mixture.
f) Allow the second centrifuge tube to stand for 60 minutes at the original temperature.
g) Immediately after step f), centrifuge the second centrifuge tube at 500 g for 10 minutes and then take another 0.05 mL subsample of the supernatant (subsample B).
h) Recover the centrifugation pellet of step g) if there is one, resuspend it in 0 water and immediately inspect the suspension for presence of crystals that are visible by microscopy.
i) Determine the concentration of TP in subsamples A and B—the results are expressed as % TP w/w relative to the total weight of the subsamples. The concentration of TP of subsample A is referred to as CTP, A and the concentration of TP of subsample B is referred to as CTP, B.
j) The liquid from which the sample of step a) was taken was supersaturated (at the specific conditions) if CTP, B is lower than CTP, A and if crystals are observed in step i).

In the context of the present invention, the terms “liquid” and “solution” encompass compositions that contain a combination of liquid and solid or semi-solid particles such as e.g. protein crystals or other protein particles. A “liquid” or a “solution” may therefore be a suspension or even a slurry. However, a “liquid” and “solution” is preferably pumpable.

In the context of the present invention, a dry product such as e.g. a powder, which comprises “TP crystals” contains the product obtained from drying a suspension of TP crystals and the crystal structure of the wet TP crystals may have been distorted during the drying process and may at least partially have lost their x-ray diffraction characteristics. Along the same lines, the terms “dry TP crystal” and “dried TP crystal” refer to the particle obtained from drying a wet TP crystal and this dry particle need not have a crystal structure itself. However, the present inventors have observed that when dried TP crystals are resuspended in cold (4 degrees C.) demineralised water in the weight ratio 2 part water to 1 part dried TP crystals the TP crystal are rehydrated and resume substantially the same crystal structure (space group-type and unit cell dimension) as before drying.

Methods of analysis described in the context of PCT Application WO 2018/115520 apply equally to the present invention and should be used for determining the parameters described herein.

In some preferred embodiments of the invention, the method does not contain the separation of step c) and provides a product, which comprises both TP crystals and the additional protein.

If this method variant furthermore include the drying of step f) it provides a dry product containing TP crystals and the additional protein. Preferably, the method contains the steps a), b) and f) in direct sequence.

In some preferred embodiments of the invention, the method furthermore comprises a step d) of washing TP crystals, e.g. the separated TP crystals obtained from step c).

In some preferred embodiments of the invention, the method furthermore comprises a step e) of re-crystallising TP crystals, e.g. the TP crystals obtained from step c) or d).

The method may e.g. comprise, or even consist of, steps a), b), c), d), and e). Alternatively, the method may comprise, or even consist, of steps a), b), c), and e).

In some particularly preferred embodiments of the invention, the method furthermore comprises a step f) of drying a TP-containing composition derived from step b), c), d), or e).

The method may for example comprise, or even consist of, steps a), b), and f).

Alternatively, the method may comprise, or even consist of, steps a), b), c) and f).

Alternatively, the method may comprise, or even consist of, steps a), b), c), d) and f).

Alternatively, the method may comprise, or even consist of, steps a), b), c), d), e) and f).

The present method preferably comes with the proviso that the TP is not a beta-lactoglobulin and/or has a sequence identity relative to bovine BLG of at most 50%, or even more preferably at most 26%.

In the context of the present invention, the term “BLG” or “beta-lactoglobulin” pertains to BLG from mammal species, e.g. in native and/or glycosylated forms and includes the naturally occurring genetic variants. The term BLG also encompasses mammal BLG produced by recombinant microorganisms. The term “BLG” or “beta-lactoglobulin” as used herein excludes unfolded and aggregated BLG. Beta-lactoglobulin is a lipocalin protein, and can bind many hydrophobic molecules, suggesting a role in their transport. Beta-lactoglobulin has also been shown to be able to bind iron via siderophores and thus might have a role in combating pathogens. Upon ingestion BLG is able to shuttle complexed iron into human immune cells, thereby providing micronutrition to these cells and participating in immune tolerance.

The target protein preferably comprises a polypeptide sequence comprising at least 30 consecutive amino acid residues, more preferably at least 50 consecutive amino acid residues, even more preferably at least 100 consecutive amino acid residues, and more preferable at least 150 consecutive amino acid residues.

Preferably, the TP is a globular protein, and preferably a globulin or a globin.

The term “TP” or “target protein” pertains to the native target protein, which has not been heat-denatured or denatured by other means.

In some preferred embodiments of the present invention the TP is selected from the group consisting of an enzyme, an immunoglobulin, a hormone.

In some preferred embodiments of the present invention the TP is an enzyme, and preferably an oxidoreductase (EC 1), a transferase (EC 2), a hydrolase (EC 3), a lyase (EC 4), or an isomerase (EC 5).

The “EC” numbers mentioned herein refer to the “enzyme commision number” for classifying enzymes according to their catalytic functionality.

In some preferred embodiments of the present invention the TP is an oxidoreductase enzyme (EC 1), preferably selected from one or more of the following a Laccase (EC 1.10.3.2), a Catalase (EC 1.11.1.6), a Glucose oxidase (EC 1.1.3.4), a Ligninase (EC 1.11.1.14) and a Peroxidase (EC 1.11.1).

In other preferred embodiments of the present invention the TP is a transferase enzyme (EC 2), preferably selected from an acyltransferase (EC 2.3) or a glycosyltransferase (EC 2.4).

In further preferred embodiments of the present invention the TP is a hydrolase enzyme (EC 3).

In some embodiments where the TP has hydrolyses activity the TP is an esterase enzyme (EC 3.1), preferably selected from a phytase (EC 3.1.3), a lipase (EC 3.1.1.3), phosphatase (EC 3.1.3), a phytase, and/or a nuclease (EC 3.1.11-3.1.31).

It is particularly preferred that the TP is a lipase (EC 3.1.1.3) which is a class of enzymes that have may important industrial applications. Examples of lipases that are capable of crystallizing under salting-in conditions are e.g. found in U.S. Pat. No. 5,719,048 which is incorporated herein by reference.

In other embodiments where the TP has hydrolyses activity the TP is a glycosylase enzyme (EC 3.2), preferably selected from an alpha-amylase (EC 3.2.1.1), an Amyloglucosidase (EC 3.2.1.3 or EC 3.2.1.33), a Cellulase (EC 3.2.1.4), a Hemicellulase (EC 3.2.1.4), a Xylanase (EC 3.2.1.8), a Muramidase (EC 3.2.1.17), a Pullulanase (EC 3.2.1.41), a Pectinase (EC 3.2.1.67), a beta-galactosidase (EC 3.2.1.23), a lactase (EC 3.2.1.108), a Glucoamylase (EC 3.2.1.20 or EC 3.2.1.3), and a Dextranase (EC 3.2.1.11).

In further embodiments where the TP has hydrolyses activity the TP is a peptidase or protease enzyme (EC 3.4), e.g. selected from a chymotrypsin (EC 3.4.21.1), a trypsin, a pepsin, a chymosin (EC 3.4.23.4), or an alkaline protease.

Alternatively, TP may be a lyase enzyme (EC 4), e.g. a pectate lyase or a alpha-acetolactate decarboxylase.

In other preferred embodiments where the TP is an isomerase enzyme (EC 5), e.g. a glucose isomerase.

Alternatively, but also preferred, the TP may be a protein hormone, preferably human insulin (uniprot ID: P01308), a human insulin analogue, a human insulin agonist, human GLP-1, a human GLP-1 analogue, or a human GLP-1 agonist,.

In the context of the present invention, the term “an analogue” used in a relation to a first protein hormone pertains to a second protein that has a sequence identity of at least 90% relative to the first protein hormone and which has the same or a similar hormonal functionality as the first protein hormone. The determination of sequence identity is based on the mature proteins and therefore disregards signal peptides.

In the context of the present invention the term “sequence identity” pertains to the relatedness between two amino acid sequences. The sequence identity between two amino acid sequences is determined using the Needleman-Wunsch algorithm (Needleman and Wunsch, 1970, J. Mol. Biol. 48:443-453) as implemented in the Needle program of the EMBOSS package (EMBOSS: The European Molecular Biology Open Software Suite, Rice et al., 2000, Trends Genet. 16:276-277), preferably version 5.0.0 or later. The parameters used are a gap open penalty of 10, a gap extension penalty of 0.5, and the EBLOSUM62 (EMBOSS version of BLOSUM62) substitution matrix. The output of Needle labeled “longest identity” (obtained using the-nobrief option) is used as the percent identity and is calculated as follows:

(Identical Residues×100)/(Length of Alignment−Total Number of Gaps in Alignment)

A further type of useful TP is an immunoglobulin, and preferably a monoclonal immunoglobulin. The TP may be any type of immunoglobulin, however, immunoglobulin G (IgG) is presently preferred, and preferably in the form of monoclonal IgG.

In some preferred embodiments of the invention TP is a bispecific IgG.

In other preferred embodiments of the invention the TP is an interferon, an interferon analogue, or an interferon agonist.

Useful examples of therapeutic proteins which are immunoglobulins or derived from immunoglobulin which can be found in Lu et al.; Development of therapeutic antibodies for the treatment of diseases. J Biomed Sci 27, 1 (2020). https://doi.org/10.1186/s12929-019-0592-z which is incorporated herein for all purposes.

The TP may be prepared by purification from a natural source, by fermentation in recombinant organisms, and/or by subsequent protein rearrangement.

The TP as such may be a recombinant protein, and e.g. a fusion protein, or a protein found in nature or in the human body.

It is particularly preferred that the TP is a therapeutic protein, i.e. a protein having a therapeutic effect. Useful examples of TPs having therapeutic effects are e.g. found in Lagasse et al (“Recent advances in (therapeutic protein) drug development” [version 1; referees: 2 approved] F1000Research 2017, 6 (F1000 Faculty Rev): 113 (doi: 10.12688/f1000research.9970.1)) and/or in Leader et al (“Protein therapeutics: a summary and pharmacological classification”; Nature Reviews Drug Discovery; volume 7, pages21-39 (2008)) which are incorporated herein by reference.

Preferably, the TP is a therapeutic protein selected from the group consisting of MuromonabCD3, Abciximab, Rituximab, Palivizumab, Infliximab, Trastuzumab, Alemtuzumab, Adalimumab, Ibritumomab tiuxetan, Omalizumab, Cetuximab, Bevacizumab, Natalizumab, Panitumumab, Ranibizumab, Eculizumab, Certolizumab pegol, Ustekinumab, Canakinumab, Golimumab, Ofatumumab, Tocilizumab, Denosumab, Belimumab, Ipilimumab, Brentuximab vedotin, Pertuzumab, Trastuzumab emtansine, Raxibacumab, Obinutuzumab, Siltuximab, Ramucirumab, Vedolizumab, Blinatumomab, Nivolumab, Pembrolizumab, Idarucizumab, Necitumumab, Dinutuximab, Secukinumab, Mepolizumab, Alirocumab, Evolocumab, Daratumumab, Elotuzumab, Ixekizumab, Reslizumab, Olaratumab, Bezlotoxumab, Atezolizumab, Obiltoxaximab, Inotuzumab ozogamicin, Brodalumab, Guselkumab, Dupilumab, Sarilumab, Avelumab, Ocrelizumab, Emicizumab, Benralizumab, Gemtuzumab ozogamicin, Durvalumab, Burosumab, Lanadelumab, Mogamulizumab, Erenumab, Galcanezumab, Tildrakizumab, Cemiplimab, Emapalumab, Fremanezumab, Ibalizumab, Moxetumomab pasudodox, Ravulizumab, Caplacizumab, Romosozumab, Risankizumab, Polatuzumab vedotin, Brolucizumab, and Crizanlizumab.

The skilled person knows how to quantify the amount of TP, and will typically use a method based on chromatography, preferably HPLC, in combination with a suitable detection method.

Step a) involves preparing an initial protein solution comprising the TP, said initial protein solution is supersaturated with respect to the TP and has a pH at which the TP is capable of crystalizing under salting-in conditions, preferably in the range of +/−0.7 pH-units of the pH that provides the maximum crystallisation yield of the TP (pH_{MCY,TP}), and most preferably in the range of +/−0.5 pH-units of the pH_{MCY,TP}.

The initial protein solution of step a) is prepared by combining one or more type A protein feed(s) with one or more type B protein feed(s), preferably by mixing the one or more type A protein feed(s) with the one or more type B protein feed(s).

In the context of the present invention a “type A protein feed” is defined as a protein feed which comprises the TP and has a pH that is at least 0.1 pH unit higher than the pH_{MCY,TP}. Any TP-containing protein feed which has a pH that is at least 0.1 pH unit higher than the pH_{MCY,TP} and is used for preparing the initial protein solution is therefore considered a type A protein feed.

In the context of the present invention a “type B protein feed” is defined as a protein feed which comprises the TP and has a pH that is at least 0.1 pH unit lower than the pH_{MCY,TP}. Any TP-containing protein feed which has a pH that is at least 0.1 pH unit lower than the pH_{MCY,TP} and is used for preparing the initial protein solution is therefore considered a type B protein feed.

In the context of the present invention the term “initial protein solution” pertains to the protein solution prepared by combining, and preferably mixing, one or more type A protein feed(s) and one or more type B protein feed(s). The initial protein solution is the TP-containing solution which is supersaturated with respect to TP and in which the crystallisation of step b) is initiated. If one or more of the used protein feed(s) already contain TP crystals the initial protein solution is the first protein solution which has a pH in the range of +/−0.7 pH-units of pH_{MCY,TP}, is supersaturation with respect to the TP, and which is obtained by combining, and preferably mixing, a Type A protein feed and a Type B protein feed.

In the context of the present invention the term “one or more type A protein feed(s)” means that the invention may be implemented with a single type A protein feed or with several type A protein feeds which are combined with the type B protein feed(s) during the preparation of the initial protein solution.

In the context of the present invention the term “one or more type B protein feed(s)” means that the invention both may be implemented with a single type B protein feed or with several type B protein feeds which are combined with the type A protein feed(s) during the preparation of the initial protein solution.

In the context of the present invention the term “the type A protein feed(s) would, if combined, . . . ” describes the one or more type A protein feed(s) based on the characteristics they would have had if they hypothetically were mixed to a single type A protein feed in the same amounts as they are used to prepare the initial protein solution. It is important to note that this term also encompasses the use of only a single type A protein feed in which case the characteristics relate to the single type A protein feed. It is furthermore important to note that this term does not require that multiple type A protein feeds are actually combined prior to mixing with the one or more type B protein feed(s).

In the context of the present invention the term “the type B protein feed(s) would, if combined, . . . ” describes the one or more type B protein feed(s) based on the characteristics they would have had if they hypothetically were mixed to a single type B protein feed in the same amounts as they are used to prepare the initial protein solution. It is important to note that this term also encompasses the use of only a single type B protein feed in which case the characteristics relate to the single type B protein feed. It is furthermore important to note that this term does not require that multiple type B protein feeds are actually combined prior to mixing with the one or more type A protein feed(s).

The initial protein solution of step a) typically comprises non-TP solids in addition to TP. In the context of the present invention the term “non-TP solids” pertains to solids such as e.g. carbohydrates, minerals, lipids, peptides as well as other proteins than TP. The solids present in the initial protein solution are characteristic for the protein source or sources from which the protein feeds have been prepared. Protein feeds based on TP sources prepared by fermentation typically contain impurities from the fermentation broth e.g. residual nutrients and metabolic byproducts such as e.g. other proteins, peptides and metabolized nutrients.

The initial protein solution often contain additional protein in addition to TP. In the context of the present invention, the term “additional protein” means a protein that is not TP. The additional protein is typically protein impurities or cell debris from a fermentation broth, e.g. if the TP has been produced by fermentation of a recombinant cell culture.

In some preferred embodiments of the present invention the initial protein solution of step a) comprises non-TP solids in an amount of at least 1% w/w relative to total solids, more preferably at least 2% w/w, even more preferred at least 5% w/w and most preferred at least 10% w/w.

In other preferred embodiments of the present invention the initial protein solution of step a) comprises non-TP solids in an amount of at least 15% w/w relative to total solids, more preferably at least 20% w/w, even more preferred at least 30% w/w and most preferred at least 40% w/w.

In some preferred embodiments of the present invention the initial protein solution of step a) comprises non-TP solids in an amount of 0-70% w/w relative to total solids, more preferably 1-60% w/w, even more preferred 2-50% w/w and most preferred 3-40% w/w.

In other preferred embodiments of the present invention the initial protein solution of step a) comprises non-TP solids in an amount of 0-20% w/w relative to total solids, more preferably 0-15% w/w, even more preferred 0-10% w/w and most preferred 0-5% w/w.

In further preferred embodiments of the present invention the initial protein solution of step a) comprises non-TP solids in an amount of 15-70% w/w relative to total solids, more preferably 20-65% w/w, even more preferred 25-60% w/w and most preferred 30-50% w/w.

In some preferred embodiments of the invention, the initial protein solution of step a) comprises at least 5% w/w additional protein relative to the total amount of protein. Preferably, the initial protein solution of step a) comprises at least 10% w/w additional protein relative to the total amount of protein. More preferably, the initial protein solution of step a) comprises at least 15% w/w additional protein relative to the total amount of protein. Even more preferably, the initial protein solution of step a) comprises at least 20% w/w additional protein relative to the total amount of protein. Most preferably, the initial protein solution of step a) may comprise at least 30% w/w additional protein relative to the total amount of protein.

In yet other preferred embodiments of the invention, the initial protein solution of step a) comprises at least 35% w/w additional protein relative to the total amount of protein. Preferably, the initial protein solution of step a) may comprise at least 40% w/w additional protein relative to the total amount of protein. More preferably, the initial protein solution of step a) may e.g. comprise at least 45% w/w additional protein relative to the total amount of protein. Even more preferably, the initial protein solution of step a) may comprise at least 50% w/w additional protein relative to the total amount of protein.

In some preferred embodiments of the invention the initial protein solution of step a) comprises in the range of 5-90% w/w additional protein relative to the total amount of protein. Preferably, the initial protein solution of step a) may comprise in the range of 10-80% w/w additional protein relative to the total amount of protein. The initial protein solution of step a) may e.g. comprise in the range of 20-70% w/w additional protein relative to the total amount of protein.

Preferably, the initial protein solution of step a) comprises in the range of 30-70% w/w additional protein relative to the total amount of protein.

As said, the present inventors have found that it is possible to crystallize TP without the use of organic solvents. This purification approach can also be used to refine preparations containing other proteins, which preparations have already been subjected to some TP purification and provides simple methods of increasing the purity of TP even further. Thus, in some preferred embodiments of the invention the initial protein solution of step a) comprises in the range of 1-20% w/w additional protein relative to the total amount of protein. Preferably, the initial protein solution of step a) may comprise in the range of 2-15% w/w additional protein relative to the total amount of protein. Even more preferably, the initial protein solution of step a) may e.g. comprise in the range of 3-10% w/w additional protein relative to the total amount of protein.

Amounts and concentrations of TP and other proteins in the initial protein solution and the protein feed all refer to dissolved protein and do not include precipitated or crystallised protein.

In the context of the present invention, the term “weight ratio” between component X and component Y means the value obtained by the calculation mx/my wherein mx is the amount (weight) of components X and my is the amount (weight) of components Y.

In some preferred embodiments of the invention the initial protein solution of step a) comprises at least 1% w/w TP relative to the total amount of protein. Preferably, the initial protein solution of step a) comprises at least 2% w/w TP relative to the total amount of protein. Even more preferably, the initial protein solution of step a) comprises at least 5% w/w TP relative to the total amount of protein. Preferably, the initial protein solution of step a) may comprise at least 10% w/w TP relative to the total amount of protein.

In some preferred embodiments of the invention the initial protein solution of step a) comprises at least 12% w/w TP relative to the total amount of protein. For example, the initial protein solution of step a) may comprise at least 15% w/w TP relative to the total amount of protein. The initial protein solution of step a) may e.g. comprise at least 20% w/w TP relative to the total amount of protein. Alternatively, the initial protein solution of step a) may comprise at least 30% w/w TP relative to the total amount of protein.

In other preferred embodiments of the invention the initial protein solution of step a) comprises at least 40% w/w TP relative to the total amount of protein. More preferably, the initial protein solution of step a) may comprise at least 45% w/w TP relative to the total amount of protein. Even more preferably, the initial protein solution of step a) may e.g. comprise at least 50% w/w TP relative to the total amount of protein. Most preferably, the initial protein solution of step a) may comprise at least 55% w/w TP relative to the total amount of protein.

In some particularly preferred embodiments of the invention the initial protein solution of step a) comprises at most 95% w/w TP relative to the total amount of protein. Preferably, the initial protein solution of step a) may comprise at most 90% w/w TP relative to the total amount of protein. More preferably, the initial protein solution of step a) may e.g. comprise at most 85% w/w TP relative to the total amount of protein. Even more preferably, the initial protein solution of step a) may e.g. comprise at most 80% w/w TP relative to the total amount of protein. Preferably, the initial protein solution of step a) may comprise at most 78% w/w TP relative to the total amount of protein. Preferably, the initial protein solution of step a) may comprise at most 75% w/w TP relative to the total amount of protein.

In some preferred embodiments of the invention the initial protein solution of step a) comprises in the range of 1-95% w/w TP relative to the total amount of protein. Preferably, the initial protein solution of step a) may comprise in the range of 5-90% w/w TP relative to the total amount of protein. More preferably the initial protein solution of step a) comprises in the range of 10-85% w/w TP relative to the total amount of protein. Even more preferably the initial protein solution of step a) comprises in the range of 10-80% w/w TP relative to the total amount of protein. Most preferably, the initial protein solution of step a) may comprise in the range of 20-70% w/w TP relative to the total amount of protein.

In other preferred embodiments of the invention the initial protein solution of step a) comprises in the range of 10-95% w/w TP relative to the total amount of protein. Preferably, the initial protein solution of step a) may comprise in the range of 12-90% w/w TP relative to the total amount of protein. More preferably the initial protein solution of step a) comprises in the range of 15-85% w/w TP relative to the total amount of protein. Even more preferably the initial protein solution of step a) comprises in the range of 15-80% w/w TP relative to the total amount of protein. Most preferably, the initial protein solution of step a) may comprise in the range of 30-70% w/w TP relative to the total amount of protein.

In some preferred embodiments of the invention the initial protein solution of step a) comprises TP in an amount of at least 0.4% w/w relative to the weight of the initial protein solution. Preferably the initial protein solution comprises TP in an amount of at least 1.0% w/w. More preferably the initial protein solution comprises TP in an amount of at least 2.0% w/w. It is even more preferred that the initial protein solution comprises TP in an amount of at least 4% w/w.

Higher concentrations of TP are even more preferred and preferably the initial protein solution comprises TP in an amount of at least 6% w/w. More preferably, the initial protein solution comprises TP in an amount of at least 10% w/w. It is even more preferred that the initial protein solution comprises TP in an amount of at least 15% w/w.

In some preferred embodiments of the invention the initial protein solution of step a) comprises TP in an amount in the range of 0.4-40% w/w relative to the weight of the initial protein solution. Preferably the initial protein solution comprises TP in an amount in the range of 1-35% w/w. More preferably the initial protein solution comprises TP in an amount in the range of 4-30% w/w. It is even more preferred that the initial protein solution comprises TP in an amount in the range of 10-25% w/w.

In other preferred embodiments of the invention the initial protein solution of step a) comprises TP in an amount in the range of 1-45% w/w relative to the weight of the initial protein solution. Preferably the initial protein solution comprises TP in an amount in the range of 3-40% w/w. More preferably the initial protein solution comprises TP in an amount in the range of 5-36% w/w. It is even more preferred that the initial protein solution comprises TP in an amount in the range of 7-34% w/w.

In further preferred embodiments of the invention the initial protein solution of step a) comprises TP in an amount in the range of 6-32% w/w relative to the weight of the initial protein solution. Preferably the initial protein solution comprises TP in an amount in the range of 8-30% w/w. More preferably the initial protein solution comprises TP in an amount in the range of 10-28% w/w. It is even more preferred that the initial protein solution comprises TP in an amount in the range of 12-26% w/w.

It is preferred that the initial protein solution is a demineralised initial protein solution.

In this context the term demineralised means that the conductivity of the initial protein solution is at most 15 mS/cm, and preferably at most 10 mS/cm, and even more preferably at most 8 mS/cm. The UF permeate conductivity of a demineralised initial protein solution is preferably at most 7 mS/cm, more preferably at most 4 mS/cm, and even more preferably at most 1 mS/cm.

The terms “consists essentially of” and “consisting essentially of” mean that the claim or feature in question encompasses the specified materials or steps and those that do not materially affect the basic and novel characteristic(s) of the claimed invention.

The protein of the initial protein solution is preferably as close to its native state as possible and preferably have only been subjected to gentle, non-denaturing heat-treatments if any at all.

The present method is particularly advantageous for separating TP from crude initial protein solutions that contain other solids than TP.

The initial protein solution may for example contain carbohydrates, such as e.g. sucrose, glucose, lactose, oligosaccharides and/or hydrolysis products of lactose (i.e. glucose and galactose). The initial protein solution may e.g. contain carbohydrate in the range of 0-40% w/w, such as in the range of 1-30% w/w, or in the range of 2-20% w/w.

In some preferred embodiments of the invention the initial protein solution contains at most 20% w/w carbohydrate, preferably at most 10% w/w carbohydrate, more preferably at most 5% w/w carbohydrate, and even more preferably at most 2% w/w carbohydrate.

The initial protein solution may also comprise lipid, e.g. in the form of triglyceride and/or other lipid types such as phospholipids.

In some embodiments of the invention the initial protein solution of step a) comprises a total amount of lipid of at most 15% w/w relative to total solids. Preferably, the initial protein solution of step a) comprises a total amount of lipid of at most 10% w/w relative to total solids. More preferably, the initial protein solution of step a) comprises a total amount of lipid of at most 6% w/w relative to total solids. Even more preferably, the initial protein solution of step a) comprises a total amount of lipid of at most 1.0% w/w relative to total solids. Most preferably, the initial protein solution of step a) comprises a total amount of lipid of at most 0.5% w/w relative to total solids.

The total amount of protein of the initial protein solution is typically at least 1% w/w relative to the weight of the initial protein solution. Preferably, the total amount of protein of the initial protein solution is at least 5% w/w. More preferred, the total amount of protein of the initial protein solution is at least 10% w/w. Even more preferred, the total amount of protein of the initial protein solution is at least 15% w/w.

In some preferred embodiments of the invention the total amount of protein of the initial protein solution is in the range of 1-50% w/w. Preferably, the total amount of protein of the initial protein solution is in the range of 5-40% w/w. More preferred, the total amount of protein of the initial protein solution is in range of 10-30% w/w. Even more preferred, the total amount of protein of the initial protein solution is in the range of 15-25% w/w.

The total solids of the initial protein solution is typically at least 1% w/w relative to the weight of the initial protein solution. Preferably, the total solids of the initial protein solution is at least 5% w/w. More preferred, the total solids of the initial protein solution is at least 10% w/w. Even more preferred, the total solids of the initial protein solution is at least 15% w/w.

In some preferred embodiments of the invention the total solids of the initial protein solution is in the range of 5-50% w/w. Preferably, the total solids of the initial protein solution is in the range of 8-40% w/w. More preferred, the total solids of the initial protein solution is in range of 10-30% w/w. Even more preferred, the total solids of the initial protein solution is in the range of 15-25% w/w.

In other preferred embodiments of the invention the total solids of the initial protein solution is in the range of 8-50% w/w. Preferably, the total solids of the initial protein solution is in the range of 10-45% w/w. More preferred, the total solids of the initial protein solution is in range of 12-40% w/w. Even more preferred, the total solids of the initial protein solution is in the range of 15-35% w/w.

The total amount of protein of the initial protein solution is determined according to Example 9.2 of WO2018/115520.

The initial protein solution of the step a) is prepared by combining, and preferably mixing, one or more type A protein feed(s) with one or more type B protein feed(s).

In a most preferred embodiment, the initial protein solution is prepared by combining, and preferably mixing, a single type A protein feed with a single type B protein feed. The crystallisation is preferably initiated by subsequent addition of a seed composition, preferably in the form of a liquid or alternatively in the form of a powder.

The initial protein solution may furthermore contain protein feed(s) that are neither of Type A nor B, e.g. protein feed(s) having a pH of pH_{MCY,TP} or protein feed(s) not containing TP, but it is preferred to limit or even avoid the presence of such additional protein feeds unless they are used to optimize the processes, e.g. by providing TP crystals or other seed materials to initiate crystallisation.

The preparation of the initial protein solution may involve addition of other ingredients in addition to the protein feeds, e.g. addition of acid or base in order to provide an initial protein solution with a suitable pH. However, it is preferred that the one or more type A protein feed(s) and the one or more type B protein feed(s) have already been conditioned to provide the desired pH when combined and hence to limit or even avoid addition of other ingredients.

The protein feeds are typically aqueous liquid and/or powder comprising TP and impurities. Examples of typical impurities are minerals, carbohydrates, other proteins such as e.g. additional protein.

The one or more type A protein feed(s), the one or more Type B protein feed(s), and any further ingredients may therefore be combined in any order to provide the initial protein solution.

The initial protein solution is prepared by combining the protein feeds, preferably by mixing one or more type A protein feed(s) with the one or more Type B protein feed(s), and optionally with any further protein feeds or ingredients.

All protein feeds and any further ingredients are provided with appropriate chemical compositions and combined in amounts sufficient to provide an initial protein solution as described herein.

The inventors have found that the maximum TP yield of the TP crystallisation is obtained at approximately pH_{MCY,TP}. It is therefore preferred that the initial protein solution has a pH that is closer to pH_{MCY,TP} than:
the pH of the Type A protein feed(s) if they were combined, or
the pH of the Type B protein feed(s) if they were combined, or
the pH of the Type A protein feed(s) if they were combined and the pH of the Type B protein feed(s) if they were combined.

It is often preferred to combine the protein feeds to provide an initial protein solution which is in the meta-stable region with respect to TP supersaturation, i.e. in the supersaturated region where TP crystals can grow when seeding is used but where crystallisation does not start spontaneously.

In some preferred embodiments of the present invention:
the type A protein feed(s) would, if combined, have a pH of at least pH_{MCY,TP}+0.1, more preferably at least pH_{MCY,TP}+0.2, even more preferably at least pH_{MCY,TP}+0.3, and most preferably at least pH_{MCY,TP}+0.4, and
the type B protein feed(s) would, if combined, have a pH of at most pH_{MCY,TP}−0.1, more preferably at most pH_{MCY,TP}−0.2, even more preferably at most pH_{MCY,TP}−0.3, and most preferably at most pH_{MCY,TP}−0.4.

If only a single type A protein feed is used the phase “would, if combined,” refers to the single single type A protein feed as such. Similarly, if only a single type B protein feed is used the phase “would, if combined,” refers to the single type B protein feed as such.

Therefore the phrase “the type A protein feed(s) would, if mixed, have [a specific property]” means that
if only a single type A protein feed is used it has [the specific property], or
if two or more type A protein feeds are used they would, if mixed, have [specific property].

For example, by stating that the type A protein feed(s) would, if mixed, have a pH of at least pH_{MCY,TP}+0.1, it is meant that:
if only a single type A protein feed is used it has a pH of at least pH_{MCY,TP}+0.1
if two or more type A protein feeds are used they would, if mixed, have a pH of at least pH_{MCY,TP}+0.1.

For example, in an embodiment wherein two type A protein feeds are used the phrase “would, if combined” means that the specific property would be obtained if the two type A protein feeds were mixed.

Thus, in some preferred embodiments of the present invention:
in relation to type A protein feed:
   if only a single type A protein feed is used it has, or
   if two or more type A protein feeds are used they would, if mixed, have
a pH of at least pH_{MCY,TP}+0.1, more preferably at least pH_{MCY,TP}+0.2, even more preferably at least pH_{MCY,TP}+0.3, and most preferably at least pH_{MCY,TP}+0.4, and
in relation to type B protein feed:
   if only a single type B protein feed is used it has, or
   if two or more type B protein feeds are used they would, if mixed, have
a pH of at most pH_{MCY,TP}−0.1, more preferably at most pH_{MCY,TP}−0.2, even more preferably at most pH_{MCY,TP}−0.3, and most preferably at most pH_{MCY,TP}−0.4.

Preferably:
in relation to type A protein feed:
   if only a single type A protein feed is used it has, or
   if two or more type A protein feeds are used they would, if mixed, have
a pH of at least pH_{MCY,TP}+0.5, more preferably at least pH_{MCY,TP}+0.6, even more preferably at least pH_{MCY,TP}+0.7, and most preferably at least pH_{MCY,TP}+0.8, and
in relation to type B protein feed:
   if only a single type B protein feed is used it has, or
   if two or more type B protein feeds are used they would, if mixed, have
a PH of at most pH_{MCY,TP}−0.5, more preferably at most pH_{MCY,TP}−0.6, even more preferably at most pH_{MCY,TP}−0.7, and most preferably at most pH_{MCY,TP}−0.8.

In other preferred embodiments of the present invention:
in relation to type A protein feed:
   if only a single type A protein feed is used it has, or
   if two or more type A protein feeds are used they would, if mixed, have
a pH of at least pH_{MCY,TP}+0.9, more preferably at least pH_{MCY,TP}+1.0, even more preferably at least pH_{MCY,TP}+1.2, and most preferably at least pH_{MCY,TP}+1.5, and
in relation to type B protein feed:
   if only a single type B protein feed is used it has, or
   if two or more type B protein feeds are used they would, if mixed, have
a pH of at most pH_{MCY,TP}−0.9, more preferably at most pH_{MCY,TP}−1.0, even more preferably at most pH_{MCY,TP}−1.2, and most preferably at most pH_{MCY,TP}−1.5.

Preferably, in relation to type A protein feed:
if only a single type A protein feed is used it has, or
if two or more type A protein feeds are used they would, if mixed, have a pH of in the range of pH_{MCY,TP}+0.1 to pH_{MCY,TP}+4.5, more preferably pH_{MCY,TP}+0.3 to pH_{MCY,TP}+2.5, even more preferably pH_{MCY,TP}+0.5 to pH_{MCY,TP}+1.5 and most preferably pH_{MCY,TP}+0.6 to pH_{MCY,TP}+1.0.

Alternatively, but also preferably:
if only a single type A protein feed is used it has, or
if two or more type A protein feeds are used they would, if mixed, have a pH of in the range of pH_{MCY,TP}+0.5 to pH_{MCY,TP}+2.5, more preferably pH_{MCY,TP}+0.6 to pH_{MCY,TP}+2.0, even more preferably pH_{MCY,TP}+0.7 to pH_{MCY,TP}+1.5 and most preferably pH_{MCY,TP}+0.7 to pH_{MCY,TP}+1.0.

In further preferred embodiments of the present invention, in relation to type A protein feed:
if only a single type A protein feed is used it has, or
if two or more type A protein feeds are used they would, if mixed, have a pH of in the range of pH_{MCY,TP}+0.7 to pH_{MCY,TP}+3.0, more preferably pH_{MCY,TP}+0.8 to pH_{MCY,TP}+2.5, even more preferably pH_{MCY,TP}+0.9 to pH_{MCY,TP}+2.0 and most preferably pH_{MCY,TP}+1.0 to pH_{MCY,TP}+1.5.

In some preferred embodiments of the present invention:
in relation to type B protein feed:
   if only a single type B protein feed is used it has, or
   if two or more type B protein feeds are used they would, if mixed, have a pH of in the range of pH_{MCY,TP}−3.5 to pH_{MCY,TP}−0.1, more preferably pH_{MCY,TP}−2.5 to pH_{MCY,TP}−0.3, even more preferably pH_{MCY,TP}−1.5 to pH_{MCY,TP}−0.5, and most preferably pH_{MCY,TP}−1.0 to pH_{MCY,TP}−0.6.

Preferably:
if only a single type B protein feed is used it has, or
if two or more type B protein feeds are used they would, if mixed, have a pH of in the range of pH_{MCY,TP}−1.5 to pH_{MCY,TP}−0.5, more preferably pH_{MCY,TP}−1.4 to pH_{MCY,TP}−0.6, even more preferably pH_{MCY,TP}−1.2 to pH_{MCY,TP}−0.7, and most preferably pH_{MCY,TP}−1.0 to pH_{MCY,TP}−0.7.

Alternatively, but also preferred, in relation to type B protein feed:
if only a single type B protein feed is used it has, or
if two or more type B protein feeds are used they would, if mixed, have a pH of in the range of pH_{MCY,TP}−2.0 to pH_{MCY,TP}−0.7, more preferably pH_{MCY,TP}−1.8 to pH_{MCY,TP}−0.8, even more preferably pH_{MCY,TP}−1.6 to pH_{MCY,TP}−0.9, and most preferably pH_{MCY,TP}−1.5 to pH_{MCY,TP}−1.0.

In some preferred embodiments of the present invention at least one of the one or more type A protein feed(s) is not saturated with respect to TP, more preferably none of the one or more type A protein feed(s) is not saturated with respect to TP.

In other preferred embodiments of the present invention at least one of the one or more type B protein feed(s) is not saturated with respect to TP, more preferably none of the one or more type B protein feed(s) is not saturated with respect to TP.

In further preferred embodiments of the present invention none of the type A or type B protein feed(s) are saturated with respect to TP.

In some preferred embodiments of the present invention at least one of the one or more type A protein feed(s) does not contain TP crystals, more preferably none of the one or more type A protein feed(s) contain TP crystals.

In other preferred embodiments of the present invention at least one of the one or more type B protein feed(s) do not contain TP crystals, more preferably none of the one or more type B protein feed(s) contain TP crystals.

It should be noted that even though a feed does not contain TP crystals it may still be saturated or even supersaturated with respect to TP.

In further preferred embodiments of the present invention none of the type A or type B protein feed(s) contain TP crystals.

In other preferred embodiments of the present invention none of the type A or type B protein feed(s) contain TP crystals.

In some preferred embodiments of the present invention at least one of the one or more type A protein feed(s) is not supersaturated with respect to TP, more preferably none of the one or more type A protein feed(s) are supersaturated with respect to TP.

In other preferred embodiments of the present invention at least one of the one or more type B protein feed(s) is not supersaturated with respect to TP, and more preferably none of the one or more type B protein feed(s) are supersaturated with respect to TP.

In some preferred embodiments of the present invention at least one of the one or more type A protein feed(s) of step a) are saturated with respect to TP.

In some preferred embodiments of the present invention at least one of the one or more type B protein feed(s) of step a) are saturated with respect to TP.

In some preferred embodiments of the present invention at least one of the one or more type A protein feed(s) is supersaturated with respect to TP but in the meta-stable zone wherein spontaneous crystallization does not occur.

In other preferred embodiments of the present invention at least one of the one or more type B protein feed(s) is supersaturated with respect to TP but in the meta-stable zone wherein spontaneous crystallization does not occur.

In some preferred embodiments of the present invention type A and type B protein feed(s) that do not contain TP crystals contribute with at least 1% w/w of the TP content of the initial protein solution, more preferably at least 10% w/w, even more preferably at least 20% w/w, and most preferably at least 40% w/w.

Preferably, type A and type B protein feed(s) that does not contain TP crystals contribute with at least 50% w/w of the TP content of the initial protein solution, more preferably at least 70% w/w, even more preferably at least 80% w/w, and most preferably at least 90% w/w.

In other preferred embodiments of the present invention type A and type B protein feed(s) that do not contain TP crystals contribute with at least 92% w/w of the TP content of the initial protein solution, more preferably at least 94% w/w, even more preferably at least 96% w/w, and most preferably at least 99% w/w.

For example, it may be preferred that type A and type B protein feed(s) that do not contain TP crystals contribute with 100% w/w of the TP content of the initial protein solution. This is for example typically the situation if seed material is added after the preparation of the initial protein solution.

The inventors have found that it often is desirable that at least some of the protein feeds are not supersaturated with respect to TP as this significantly reduces the risk of uncontrolled crystallation during production.

Thus, in some preferred embodiments of the present invention type A and type B protein feeds that are not supersaturated with respect to TP contribute with at least 1% w/w of the TP content of the initial protein solution, more preferably at least 10% w/w, even more preferably at least 20% w/w, and most preferably at least 40% w/w.

Preferably, type A and type B protein feeds that are not supersaturated with respect to TP contribute with at least 50% w/w of the TP content of the initial protein solution, more preferably at least 70% w/w, even more preferably at least 80% w/w, and most preferably at least 90% w/w.

In further preferred embodiments of the present invention type A and type B protein feeds that are not supersaturated with respect to TP contribute with at least 92% w/w of the TP content of the initial protein solution, more preferably at least 94% w/w, even more preferably at least 96% w/w, and most preferably at least 99% w/w.

It is often preferred that type A and type B protein feed(s) that are not supersaturated with respect to TP contribute with approximately 100% w/w of the TP content of the initial protein solution.

It is preferred to prepare, store and use the type A and type B protein feeds at temperatures that avoid protein denaturation and/or degradation.

In some preferred embodiments of the present invention the type A protein feed(s) would, if combined, have a temperature of at most 60 degrees C., more preferably at most 50 degrees C., even more preferably at most 40 degrees C. and most preferably at most 30 degrees C.

Thus, in some preferred embodiments of the present invention:
in relation to type A protein feed:
   if only a single type A protein feed is used it has, or
   if two or more type A protein feeds are used they would, if mixed, have

a temperature of at most 60 degrees C., more preferably at most 50 degrees C., even more preferably at most 40 degrees C. and most preferably at most 30 degrees C.

In other preferred embodiments of the present invention the type A protein feed(s) would, if combined, have a temperature of at most 20 degrees C., more preferably at most 15 degrees C., even more preferably at most 10 degrees C. and most preferably at most 5 degrees C.

Thus, in other preferred embodiments of the present invention:
in relation to type A protein feed:
   if only a single type A protein feed is used it has, or
   if two or more type A protein feeds are used they would, if mixed, have

a temperature of at most 20 degrees C., more preferably at most 15 degrees C., even more preferably at most 10 degrees C. and most preferably at most 5 degrees C.

In some preferred embodiments of the present invention the type B protein feed(s) would, if combined, have a temperature of at most 60 degrees C., more preferably at most 50 degrees C., even more preferably at most 40 degrees C. and most preferably at most 30 degrees C.

Thus, in some preferred embodiments of the present invention:
in relation to type B protein feed:
   if only a single type B protein feed is used it has, or
   if two or more type B protein feeds are used they would, if mixed, have
a temperature of at most 60 degrees C., more preferably at most 50 degrees C., even more preferably at most 40 degrees C. and most preferably at most 30 degrees C.

In other preferred embodiments of the present invention the type B protein feed(s) would, if combined, have a temperature of at most 20 degrees C., more preferably at most 15 degrees C., even more preferably at most 10 degrees C. and most preferably at most 5 degrees C.

Thus, in other preferred embodiments of the present invention, in relation to type B protein feed:
if only a single type B protein feed is used it has, or
if two or more type B protein feeds are used they would, if mixed, have
a temperature of at most 20 degrees C., more preferably at most 15 degrees C., even more preferably at most 10 degrees C. and most preferably at most 5 degrees C.

The inventors have found that initial protein solutions prepared from protein feeds having a low conductivity typically provide a higher yield of TP crystallisation.

In some preferred embodiments of the present invention the type A protein feed(s) would, if mixed, have a conductivity of at most 10 mS/cm, more preferably at most 7 mS/cm, even more preferably at most 5 mS/cm, and most preferably at most 4 mS/cm.

Thus, in some preferred embodiments of the present invention:
in relation to type A protein feed:
   if only a single type A protein feed is used it has, or
   if two or more type A protein feeds are used they would, if mixed, have

a conductivity of at most 10 mS/cm, more preferably at most 7 mS/cm, even more preferably at most 5 mS/cm, and most preferably at most 4 mS/cm.

In other preferred embodiments of the present invention the type A protein feed(s) would, if mixed, have a conductivity of at most 3 mS/cm, more preferably at most 2 mS/cm, even more preferably at most 1 mS/cm, and most preferably at most 0.5 mS/cm.

Thus, in other preferred embodiments of the present invention, in relation to type A protein feed:
if only a single type A protein feed is used it has, or
if two or more type A protein feeds are used they would, if mixed, have a conductivity of at most 3 mS/cm, more preferably at most 2 mS/cm, even more preferably at most 1 mS/cm, and most preferably at most 0.5 mS/cm.

In some preferred embodiments of the present invention the type B protein feed(s) would, if mixed, have a conductivity of at most 10 mS/cm, more preferably at most 7 mS/cm, even more preferably at most 5 mS/cm, and most preferably at most 4 mS/cm.

Thus, in some preferred embodiments of the present invention, in relation to type B protein feed:
if only a single type B protein feed is used it has, or
if two or more type B protein feeds are used they would, if mixed, have a conductivity of at most 10 mS/cm, more preferably at most 7 mS/cm, even more preferably at most 5 mS/cm, and most preferably at most 4 mS/cm.

In other preferred embodiments of the present invention the type B protein feed(s) would, if mixed, have a conductivity of at most 3 mS/cm, more preferably at most 2 mS/cm, even more preferably at most 1 mS/cm, and most preferably at most 0.5 mS/cm.

Thus, in other preferred embodiments of the present invention, in relation to type B protein feed:
if only a single type B protein feed is used it has, or
if two or more type B protein feeds are used they would, if mixed, have a conductivity of at most 3 mS/cm, more preferably at most 2 mS/cm, even more preferably at most 1 mS/cm, and most preferably at most 0.5 mS/cm.

The inventors have furthermore observed that a TP crystallisation yield is obtained if the ratio between the conductivity (expressed in mS/cm) and the total amount of protein (expressed in % wt. total protein relative to the total weight of the feed(s)) of the type A protein feed(s) and type B protein feed(s) is kept at or below a certain threshold.

In some preferred embodiments of the present invention the type A protein feed(s) would, if mixed, have a ratio between the conductivity and the total amount of protein of at most 0.3, more preferably at most 0.25, more preferably at most 0.20, more preferably at most 0.18, even more preferably at most 0.12, and most preferably at most 0.10.

Thus, in some preferred embodiments of the present invention:
in relation to type A protein feed:
   if only a single type A protein feed is used it has, or
   if two or more type A protein feeds are used they would, if mixed, have

a ratio between the conductivity and the total amount of protein of at most 0.3, more preferably at most 0.25, more preferably at most 0.20, more preferably at most 0.18, even more preferably at most 0.12, and most preferably at most 0.10.

In some preferred embodiments of the present invention the type B protein feed(s) would, if mixed, have a ratio between the conductivity and the total amount of protein of at most 0.3, more preferably at most 0.25, more preferably at most 0.20, more preferably at most 0.18, even more preferably at most 0.12, and most preferably at most 0.10.

Thus, in some preferred embodiments of the present invention, in relation to type B protein feed:
if only a single type B protein feed is used it has, or
if two or more type B protein feeds are used they would, if mixed, have a ratio between the conductivity and the total amount of protein of at most 0.3, more preferably at most 0.25, more preferably at most 0.20, more preferably at most 0.18, even more preferably at most 0.12, and most preferably at most 0.10.

In some preferred embodiments of the present invention the type A protein feed(s) would, if combined, comprises at least 1% w/w TP relative to the weight of the combination of type A protein feed(s), more preferably at least 3% w/w, even more preferably at least 5% w/w, and most preferably at least 7% w/w TP relative to the weight of the combination of type A protein feed(s).

Thus, in some preferred embodiments of the present invention, in relation to type A protein feed:
if only a single type A protein feed is used it comprises, or
if two or more type A protein feeds are used they would, if mixed, comprise at least 1% w/w TP relative to the total weight of type A protein feed, more preferably at least 3% w/w, even more preferably at least 5% w/w, and most preferably at least 7% w/w TP relative to the total weight of type A protein feed.

In other preferred embodiments of the present invention the type A protein feed(s) would, if combined, comprise 1-45% w/w TP relative to the weight of the combination of type A protein feed(s), more preferably 3-40% w/w, even more preferably 5-36% w/w, and most preferably 7-34% w/w TP relative to the weight of the combination of type A protein feed(s).

Thus, in other preferred embodiments of the present invention, in relation to type A protein feed:
if only a single type A protein feed is used it comprises, or
if two or more type A protein feeds are used they would, if mixed, comprise 1-45% w/w TP relative to the total weight of type A protein feed, more preferably 3-40% w/w, even more preferably 5-36% w/w, and most preferably 7-34% w/w TP relative to the total weight of type A protein feed.

In further preferred embodiments of the present invention the type A protein feed(s) would, if combined, comprise 6-32% w/w TP relative to the weight of the combination of type A protein feed(s), more preferably 8-30% w/w, even more preferably 10-28% w/w, and most preferably 12-26% w/w TP relative to the weight of the combination of type A protein feed(s).

Thus, in further preferred embodiments of the present invention, in relation to type A protein feed:
if only a single type A protein feed is used it comprises, or
if two or more type A protein feeds are used they would, if mixed, comprise 6-32% w/w TP relative to the total weight of type A protein feed, more preferably 8-30% w/w, even more preferably 10-28% w/w, and most preferably 12-26% w/w TP relative to the total weight of type A protein feed.

In some preferred embodiments of the present invention the type B protein feed(s) would, if combined, comprise at least 1% w/w TP relative to the weight of the combination of type B protein feed(s), more preferably at least 3% w/w, even more preferably at least 5% w/w, and most preferably at least 7% w/w TP relative to the weight of the combination of type B protein feed(s).

Thus, in some preferred embodiments of the present invention, in relation to type B protein feed:
if only a single type B protein feed is used it comprises, or
if two or more type B protein feeds are used they would, if mixed, comprise at least 1% w/w TP relative to the total weight of type B protein feed, more preferably at least 3% w/w, even more preferably at least 5% w/w, and most preferably at least 7% w/w TP relative to the total weight of type B protein feed.

In other preferred embodiments of the present invention the type B protein feed(s) would, if combined, comprise 1-45% w/w TP relative to the weight of the combination of type B protein feed(s), more preferably 3-40% w/w, even more preferably 5-36% w/w, and most preferably 7-34% w/w TP relative to the weight of the combination of type B protein feed(s).

Thus, in other preferred embodiments of the present invention, in relation to type B protein feed:
if only a single type B protein feed is used it comprises, or
if two or more type B protein feeds are used they would, if mixed, comprise 1-45% w/w TP relative to the total weight of type B protein feed, more preferably 3-40% w/w, even more preferably 5-36% w/w, and most preferably 7-34% w/w TP relative to the total weight of type B protein feed.

In further preferred embodiments of the present invention the type B protein feed(s) would, if combined, comprise 6-32% w/w TP relative to the weight of the combination of type B protein feed(s), more preferably 8-30% w/w, even more preferably 10-28% w/w, and most preferably 12-26% w/w TP relative to the weight of the combination of type B protein feed(s).

Thus, in further preferred embodiments of the present invention, in relation to type B protein feed:
if only a single type B protein feed is used it comprises, or
if two or more type B protein feeds are used they would, if mixed, comprise 6-32% w/w TP relative to the total weight of type B protein feed, more preferably 8-30% w/w, even more preferably 10-28% w/w, and most preferably 12-26% w/w TP relative to the total weight of type B protein feed.

In some preferred embodiments of the present invention the type A protein feed(s) would, if mixed, comprise TP in an amount of at least 30% w/w relative to the total amount of protein, more preferably at least 40% w/w, even more preferred at least 50% w/w and most preferred at least 55% w/w.

Thus, in some preferred embodiments of the present invention, in relation to type A protein feed:
if only a single type A protein feed is used it comprises, or
if two or more type A protein feeds are used they would, if mixed, comprise TP in an amount of at least 30% w/w relative to the total amount of protein, more preferably at least 40% w/w, even more preferred at least 50% w/w, and most preferred at least 55% w/w.

In some preferred embodiments of the present invention the type B protein feed(s) would, if mixed, comprise TP in an amount of at least 30% w/w relative to the total amount of protein, more preferably at least 40% w/w, even more preferred at least 50% w/w and most preferred at least 55% w/w.

Thus, in some preferred embodiments of the present invention, in relation to type B protein feed:
if only a single type B protein feed is used it comprises, or
if two or more type B protein feeds are used they would, if mixed, comprise TP in an amount of at least 30% w/w relative to the total amount of protein, more preferably at least 40% w/w, even more preferred at least 50% w/w, and most preferred at least 55% w/w.

In some preferred embodiments of the present invention the type A protein feed(s) would, if combined, comprise non-TP solids in an amount of at least 1% w/w relative to total solids, more preferably at least 2% w/w, even more preferred at least 5% w/w and most preferred at least 10% w/w.

Thus, in some preferred embodiments of the present invention, in relation to type A protein feed:
if only a single type A protein feed is used it comprises, or
if two or more type A protein feeds are used they would, if mixed, comprise non-TP solids in an amount of at least 1% w/w relative to total solids, more preferably at least 2% w/w, even more preferred at least 5% w/w, and most preferred at least 10% w/w.

In other preferred embodiments of the present invention the type A protein feed(s) would, if mixed, comprise non-TP solids in an amount of at least 15% w/w relative to total solids, more preferably at least 20% w/w, even more preferred at least 30% w/w and most preferred at least 40% w/w.

Thus, in other preferred embodiments of the present invention, in relation to type A protein feed:
if only a single type A protein feed is used it comprises, or
if two or more type A protein feeds are used they would, if mixed, comprise non-TP solids in an amount of at least 15% w/w relative to total solids, more preferably at least 20% w/w, even more preferred at least 30% w/w and most preferred at least 40% w/w.

In some preferred embodiments of the present invention the type A protein feed(s) would, if combined, comprise non-TP solids in an amount of 0-70% w/w relative to total solids, more preferably 1-60% w/w, even more preferred 2-50% w/w and most preferred 3-40% w/w.

Thus, in some preferred embodiments of the present invention, in relation to type A protein feed:
if only a single type A protein feed is used it comprises, or
if two or more type A protein feeds are used they would, if mixed, comprise non-TP solids in an amount of 0-70% w/w relative to total solids, more preferably 1-60% w/w, even more preferred 2-50% w/w and most preferred 3-40% w/w.

In some preferred embodiments of the present invention the type A protein feed(s) would, if mixed, comprise non-TP solids in an amount of 0-70% w/w relative to total solids, more preferably 0-60% w/w, even more preferred 0-50% w/w and most preferred 0-40% w/w.

Thus, in some preferred embodiments of the present invention, in relation to type A protein feed:
if only a single type A protein feed is used it comprises, or
if two or more type A protein feeds are used they would, if mixed, comprise non-TP solids in an amount of 0-70% w/w relative to total solids, more preferably 1-60% w/w, even more preferred 2-50% w/w and most preferred 3-40% w/w.

In other preferred embodiments of the present invention the type A protein feed(s) would, if combined, comprise non-TP solids in an amount of 0-20% w/w relative to total solids, more preferably 0-15% w/w, even more preferred 0-10% w/w and most preferred 0-5% w/w.

Thus, in other preferred embodiments of the present invention, in relation to type A protein feed:
if only a single type A protein feed is used it comprises, or
if two or more type A protein feeds are used they would, if mixed, comprise non-TP solids in an amount of 0-20% w/w relative to total solids, more preferably 0-15% w/w, even more preferred 0-10% w/w and most preferred 0-5% w/w.

In further preferred embodiments of the present invention the type A protein feed(s) would, if combined, comprise non-TP solids in an amount of 15-70% w/w relative to total solids, more preferably 20-65% w/w, even more preferred 25-60% w/w and most preferred 30-50% w/w.

Thus, in further preferred embodiments of the present invention, in relation to type A protein feed:
if only a single type A protein feed is used it comprises, or
if two or more type A protein feeds are used they would, if mixed, comprise non-TP solids in an amount of 15-70% w/w relative to total solids, more preferably 20-65% w/w, even more preferred 25-60% w/w and most preferred 30-50% w/w.

In some preferred embodiments of the present invention the type B protein feed(s) would, if combined, comprise non-TP solids in an amount of at least 1% w/w relative to total solids, more preferably at least 2% w/w, even more preferred at least 5% w/w and most preferred at least 10% w/w.

Thus, in some preferred embodiments of the present invention, in relation to type B protein feed:
if only a single type B protein feed is used it comprises, or
if two or more type B protein feeds are used they would, if mixed, comprise non-TP solids in an amount of at least 1% w/w relative to total solids, more preferably at least 2% w/w, even more preferred at least 5% w/w, and most preferred at least 10% w/w.

In other preferred embodiments of the present invention the type B protein feed(s) would, if combined, comprise non-TP solids in an amount of at least 15% w/w relative to total solids, more preferably at least 20% w/w, even more preferred at least 30% w/w and most preferred at least 40% w/w.

Thus, in other preferred embodiments of the present invention, in relation to type B protein feed:
if only a single type B protein feed is used it comprises, or
if two or more type B protein feeds are used they would, if mixed, comprise non-TP solids in an amount of at least 15% w/w relative to total solids, more preferably at least 20% w/w, even more preferred at least 30% w/w and most preferred at least 40% w/w.

In some preferred embodiments of the present invention the type B protein feed(s) would, if combined, comprise non-TP solids in an amount of 0-70% w/w relative to total solids, more preferably 1-60% w/w, even more preferred 2-50% w/w and most preferred 3-40% w/w.

Thus, in some preferred embodiments of the present invention, in relation to type B protein feed:
if only a single type B protein feed is used it comprises, or
if two or more type B protein feeds are used they would, if mixed, comprise non-TP solids in an amount of 0-70% w/w relative to total solids, more preferably 1-60% w/w, even more preferred 2-50% w/w and most preferred 3-40% w/w.

In other preferred embodiments of the present invention the type B protein feed(s) would, if combined, comprise non-TP solids in an amount of 0-20% w/w relative to total solids, more preferably 0-15% w/w, even more preferred 0-10% w/w and most preferred 0-5% w/w.

Thus, in other preferred embodiments of the present invention, in relation to type B protein feed:
if only a single type B protein feed is used it comprises, or
if two or more type B protein feeds are used they would, if mixed, comprise non-TP solids in an amount of 0-20% w/w relative to total solids, more preferably 0-15% w/w, even more preferred 0-10% w/w and most preferred 0-5% w/w.

In further preferred embodiments of the present invention the type B protein feed(s) would, if combined, comprise non-TP solids in an amount of 15-70% w/w relative to total solids, more preferably 20-65% w/w, even more preferred 25-60% w/w and most preferred 30-50% w/w.

Thus, in further preferred embodiments of the present invention, in relation to type B protein feed:
if only a single type B protein feed is used it comprises, or
if two or more type B protein feeds are used they would, if mixed, comprise non-TP solids in an amount of 15-70% w/w relative to total solids, more preferably 20-65% w/w, even more preferred 25-60% w/w and most preferred 30-50% w/w.

In some embodiments of the invention the protein feeds have low content of denatured protein. Preferably, the protein feeds would, if combined, have a degree of protein denaturation of at most 20%, more preferably at most 10%, even more preferably at most 5%, and most preferably at most 2%.

In some preferred embodiments of the present invention the initial protein solution comprises a total amount of type A protein feed in the range of 1-99% w/w relative to the weight of the initial protein solution, more preferably 10-80% w/w, even more preferably 20-70% w/w, and most preferably 30-60% w/w.

In other preferred embodiments of the present invention the initial protein solution comprises a total amount of type A protein feed in the range of 5-95% w/w relative to the weight of the initial protein solution, more preferably 15-80% w/w, even more preferably 20-75% w/w, and most preferably 35-70% w/w.

In some preferred embodiments of the present invention the initial protein solution comprises a total amount of type B protein feed(s) in the range of 1-99% w/w relative to the weight of the initial protein solution, more preferably 10-80% w/w, even more preferably 20-70% w/w, and most preferably 30-60% w/w.

In other preferred embodiments of the present invention the initial protein solution comprises a total amount of type B protein feed in the range of 5-95% w/w relative to the weight of the initial protein solution, more preferably 15-80% w/w, even more preferably 20-75% w/w, and most preferably 35-70% w/w.

In some preferred embodiments of the present invention the initial protein solution comprises:
a total amount of type A protein feed(s) in the range of 5-90% w/w relative to the weight of the initial protein solution, more preferably 10-80% w/w, even more preferably 20-70% w/w, and most preferably 30-60% w/w, and
a total amount of type B protein feed(s) in the range of 5-90% w/w relative to the weight of the initial protein solution, more preferably 10-80% w/w, even more preferably 20-70% w/w, and most preferably 30-60% w/w.

In other preferred embodiments of the present invention the initial protein solution comprises:
a total amount of type A protein feed(s) having a pH of pH_{MCY,TP}+0.3 to pH_{MCY,TP}+3.5 in the range of 5-90% w/w relative to the weight of the initial protein solution, more preferably 10-80% w/w, even more preferably 20-70% w/w, and most preferably 30-60% w/w, and
a total amount of type B protein feed(s) having a pH of pH_{MCY,TP}−3.0 to pH_{MCY,TP}−0.3 in the range of 5-90% w/w relative to the weight of the initial protein solution, more preferably 10-80% w/w, even more preferably 20-70% w/w, and most preferably 30-60% w/w.

In further preferred embodiments of the present invention the initial protein solution comprises:
a total amount of type A protein feed(s) having a pH of pH_{MCY,TP}+0.5 to pH_{MCY,TP}+2.5 in the range of 5-90% w/w relative to the weight of the initial protein solution, more preferably 10-80% w/w, even more preferably 20-70% w/w, and most preferably 30-60% w/w, and
a total amount of type B protein feed(s) having a pH of pH_{MCY,TP}−2.5 to pH_{MCY,TP}−0.5 in the range of 5-90% w/w relative to the weight of the initial protein solution, more preferably 10-80% w/w, even more preferably 20-70% w/w, and most preferably 30-60% w/w.

In even further preferred embodiments of the present invention the initial protein solution comprises:
a total amount of type A protein feed(s) having a pH of pH_{MCY,TP}+0.6 to pH_{MCY,TP}+1.5 in the range of 5-90% w/w relative to the weight of the initial protein solution, more preferably 10-80% w/w, even more preferably 20-70% w/w, and most preferably 30-60% w/w, and
a total amount of type B protein feed(s) having a pH of pH_{MCY,TP}−1.5 to pH_{MCY,TP}−0.6 of 5-90% w/w relative to the weight of the initial protein solution, more preferably 10-80% w/w, even more preferably 20-70% w/w, and most preferably 30-60% w/w.

In other preferred embodiments of the present invention the initial protein solution comprises:
a total amount of type A protein feed(s) having a pH of pH_{MCY,TP}+0.8 to pH_{MCY,TP}+1.5 in the range of 5-90% w/w relative to the weight of the initial protein solution, more preferably 10-80% w/w, even more preferably 20-70% w/w, and most preferably 30-60% w/w, and
a total amount of type B protein feed(s) having a pH of pH_{MCY,TP}−1.5 to pH_{MCY,TP}−0.8 in the range of 5-90% w/w relative to the weight of the initial protein solution, more preferably 10-80% w/w, even more preferably 20-70% w/w, and most preferably 30-60% w/w.

In some preferred embodiments of the present invention all protein feeds are in liquid form.

In other preferred embodiments of the present invention at least one of the protein feeds is in dry form.

In further preferred embodiments of the present invention at least one of the protein feeds is in dry form, and preferably in powder form and at least one of the protein feeds is in liquid form.

The embodiments relating to the chemical composition of the initial protein solution equally apply to the Type A protein feed(s) and the type B protein feed(s), however typically at least one parameter of the protein feeds is set to avoid supersaturation or at least spontaneous crystallisation.

It is often preferred that the degree of supersaturation of TP of the initial protein solution is higher than the degree of supersaturation of:
the type A protein feed if only a single type A protein feed is used, or
the type A protein feeds, if combined, if two or more type A protein feeds are used.

Additionally, it is often preferred that the degree of supersaturation of TP of the initial protein solution is higher than the degree of supersaturation of:
the type B protein feed if only a single type B protein feed is used, or
the type B protein feeds, if combined, if two or more type B protein feeds are used.

It is particularly preferred that the degree of supersaturation of TP of the initial protein solution is higher than both the degree of supersaturation of:
the type A protein feed if only a single type A protein feed is used, or
the type A protein feeds, if combined, if two or more type A protein feeds are used, and of:
the type B protein feed if only a single type B protein feed is used, or
the type B protein feeds, if combined, if two or more type B protein feeds are used.

The degree of supersaturation of a liquid is determined by seeding a sample of the liquid with TP crystals and quantifying the amount of additional TP crystals mass that is generated in the sample when keeping the sample the same conditions (e.g. temperature, pressure and humidity) for 24 hours. Quantification of TP crystal mass is done by centrifugation at 15000 g for 5 minutes at the temperature of the liquid and subsequently quantifying the content of TP in the obtained pellet using the Analysis 9.9 of WO 2018/115520A1. The degree of supersaturation is the percentage of TP of the liquid sample that is capable of being isolated in the pellet.

In some preferred embodiments of the present invention, in relation to type A protein feed, if only a single type A protein feed is used it has, or if two or more type A protein feeds are used they would, if mixed, have:
a content of TP of 4-32% w/w relative to the total weight of type A protein feed, more preferably 6-32% w/w, more preferably 8-30% w/w, even more preferably 10-28% w/w, and most preferably 12-26% w/w TP relative to the total weight of type A protein feed,
a pH of in the range of pH_{MCY,TP}+0.1 to pH_{MCY,TP}+3.5, more preferably pH_{MCY,TP}+0.3 to pH_{MCY,TP}+2.5, even more preferably pH_{MCY,TP}+0.5 to pH_{MCY,TP}+1.5 and most preferably pH_{MCY,TP}+0.6 to pH_{MCY,TP}+1.5,
preferably, a temperature of at most 20 degrees C., more preferably at most 15 degrees C., even more preferably at most 10 degrees C. and most preferably at most 5 degrees C.,
preferably, a content of TP in an amount of at least 30% w/w relative to the total amount of protein, more preferably at least 40% w/w, even more preferred at least 50% w/w, and most preferred at least 55% w/w,
one or more of:
   a conductivity of at most 7 mS/cm, even more preferably at most 5 mS/cm, and most preferably at most 3 mS/cm, and
   a ratio between the conductivity and the total amount of protein of at most 0.3, more preferably at most 0.25, most preferably at most 0.20.

In some preferred embodiments of the present invention, in relation to type B protein feed, if only a single type B protein feed is used it has, or if two or more type B protein feeds are used they would, if mixed, have:
a content of TP of 4-32% w/w relative to the total weight of type B protein feed, more preferably 6-32% w/w, more preferably 8-30% w/w, even more preferably 10-28% w/w, and most preferably 12-26% w/w TP relative to the total weight of type B protein feed,
a pH of in the range of pH_{MCY,TP}−3.5 to pH_{MCY,TP}−0.1, more preferably pH_{MCY,TP}−2.5 to pH_{MCY,TP}−0.3, even more preferably pH_{MCY,TP}−1.5 to pH_{MCY,TP}−0.5 and most preferably pH_{MCY,TP}−1.5 to pH_{MCY,TP}−0.6,
preferably, a temperature of at most 20 degrees C., more preferably at most 15 degrees C., even more preferably at most 10 degrees C. and most preferably at most 5 degrees C.,
preferably, a content of TP in an amount of at least 30% w/w relative to the total amount of protein, more preferably at least 40% w/w, even more preferred at least 50% w/w, and most preferred at least 55% w/w,
one or more of:
   a conductivity of at most 7 mS/cm, even more preferably at most 5 mS/cm, and most preferably at most 3 mS/cm, and
   a ratio between the conductivity and the total amount of protein of at most 0.3, more preferably at most 0.25, most preferably at most 0.20.

In particularly preferred embodiments of the invention,
in relation to type A protein feed, if only a single type A protein feed is used it has, or if two or more type A protein feeds are used they would, if mixed, have:
   a content of TP of 4-32% w/w relative to the total weight of type A protein feed, more preferably 6-32% w/w, more preferably 8-30% w/w, even more preferably 10-28% w/w, and most preferably 12-26% w/w TP relative to the total weight of type A protein feed,
   a pH of in the range of of pH_{MCY,TP}+0.1 to pH_{MCY,TP}+3.5, more preferably pH_{MCY,TP}+0.3 to pH_{MCY,TP}+2.5, even more preferably pH_{MCY,TP}+0.5 to pH_{MCY,TP}+1.5 and most preferably pH_{MCY,TP}+0.6 to pH_{MCY,TP}+1.5,
   preferably, a temperature of at most 20 degrees C., more preferably at most 15 degrees C., even more preferably at most 10 degrees C. and most preferably at most 5 degrees C.,
   preferably, a content of TP in an amount of at least 30% w/w relative to the total amount of protein, more preferably at least 40% w/w, even more preferred at least 50% w/w, and most preferred at least 55% w/w,
   one or more of:
      a conductivity of at most 7 mS/cm, even more preferably at most 5 mS/cm, and most preferably at most 3 mS/cm, and
      a ratio between the conductivity and the total amount of protein of at most 0.3, more preferably at most 0.25, most preferably at most 0.20.
and
in relation to type B protein feed, if only a single type B protein feed is used it has, or if two or more type B protein feeds are used they would, if mixed, have:
   a content of TP of 4-32% w/w relative to the total weight of type B protein feed, more preferably 6-32% w/w, more preferably 8-30% w/w, even more preferably 10-28% w/w, and most preferably 12-26% w/w TP relative to the total weight of type B protein feed,
   a pH of in the range of pH_{MCY,TP}−3.5 to pH_{MCY,TP}−0.1, more preferably pH_{MCY,TP}−2.5 to pH_{MCY,TP}−0.3, even more preferably pH_{MCY,TP}−1.5 to pH_{MCY,TP}−0.5 and most preferably pH_{MCY,TP}−1.5 to pH_{MCY,TP}−0.6,
   preferably, a temperature of at most 20 degrees C., more preferably at most 15 degrees C., even more preferably at most 10 degrees C. and most preferably at most 5 degrees C.,
   preferably, a content of TP in an amount of at least 30% w/w relative to the total amount of protein, more preferably at least 40% w/w, even more preferred at least 50% w/w, and most preferred at least 55% w/w,
   one or more of:
      a conductivity of at most 7 mS/cm, more preferably at most 5 mS/cm, and most preferably at most 3 mS/cm, and
      a ratio between the conductivity and the total amount of protein of at most 0.3, more preferably at most 0.25, most preferably at most 0.20.

In some preferred embodiments of the invention the protein feeds are prepared by one or more of the following process steps:
Adjusting the pH,
Reducing the conductivity
Reducing the temperature
Increasing the protein concentration
Adding an agent that reduces the water activity
Modifying the ion composition

The present inventors have found that pressure-driven membrane filtration is well-suited for preparing liquid protein feeds.

In some preferred embodiments of the present invention the at least one of the protein feeds are prepared by a process that involves pressure-driven membrane filtration of a protein source. Preferred pressure-driven membrane filtration comprises filtration by ultrafiltration, microfiltration, nanofiltration and reverse osmosis. Ultrafiltration has been found to be particularly preferred for preparing the protein feeds and may be used as the only type of pressure-driven membrane filtration during the preparation of the protein feeds or may be used in combination with the other filtration methods.

The preparation of the protein feeds often involve pH adjustments to provide protein feeds with the desired pH. Preferably the pH is adjusted to a pH in the range of pH_{MCY,TP}+0.5 to pH_{MCY,TP}+1.5 when preparing type A protein feeds and to a pH in the range of pH_{MCY,TP}−1.5 to pH_{MCY,TP}−0.5 when preparing type B protein feeds. The pH is preferably adjusted using food acceptable acids and/or bases. Food acceptable acids are particularly preferred, such as e.g. carboxylic acids. Useful examples of such acids are e.g. hydrochloric acid, sulfuric acid, phosphoric acid, acetic acid, maleic acid, tartaric acid, lactic acid, citric acid, or gluconic acid, and/or mixtures thereof.

In some preferred embodiments of the invention the pH is adjusted using a lactone, such as e.g. D-glucono-delta-lactone, which slowly hydrolyses and at the same time reduces the pH of the aqueous liquid containing it. The target pH after the hydrolysis of the lactone has ended can be calculated precisely.

Useful examples of food acceptable bases are e.g. hydroxide sources such as e.g. sodium hydroxide, potassium hydroxide, calcium hydroxide, salts of food acids such as e.g. tri-sodium citrate, and/or combinations thereof.

In other preferred embodiments of the invention the pH is adjusted by addition of cation exchange material on its H⁺ form. Bead-type/large particle type cation exchange material is easily removed from the initial protein solution prior to the crystallisation or even after the crystallisation. Adjustment of pH by addition of cation exchange material on its H⁺ form is particularly advantageous in the present invention as it reduced the pH without adding negative counter ions that significantly affects the conductivity of the protein feed.

In some preferred embodiments of the invention the preparation of the initial protein solution involves reducing the conductivity of the protein feed.

Conductivity values mentioned herein have been normalised to 25 degrees C. unless it is specified otherwise.

The inventors have found that reducing the conductivity of the initial protein solution leads to a higher yield of TP crystals. The minimum obtainable conductivity of the initial protein solution depends on the composition of the protein fraction and the lipid fraction (if any). Some protein species contribute more to the conductivity than other protein species. It is therefore preferable that the conductivity of the protein feed is brought near the level where protein and the counter ions of the protein are the main contributors to the conductivity. The reduction of conductivity often involves removal of at least some of the small, free ions that are present in liquid phase and not tightly bound to the proteins.

It is often preferred that the protein feeds have a conductivity of at most 10 mS/cm. In some preferred embodiments of the invention, the protein feeds have a conductivity of at most 5 mS/cm. Preferably, the protein feeds have a conductivity of at most 4 mS/cm.

Lower conductivities are even more preferred and give rise to higher yields of TP crystals. Thus, the protein feeds preferably have a conductivity of at most 3 mS/cm. In some preferred embodiments of the invention, the protein feeds have a conductivity of at most 1 mS/cm. Preferably, the protein feeds have a conductivity of at most 0.5 mS/cm.

The low conductivity of the protein feeds is preferably obtained by dialysis or diafiltration. Diafiltration by ultrafiltration is particularly preferred as it allows for washing out salts and small charged molecules while proteins are retained. In some preferred embodiments of the invention, the same UF unit is used for UF/diafiltration and subsequent concentration of the protein feeds.

The present inventors have seen indications that the ratio between the conductivity (expressed in mS/cm) and the total amount of protein in the initial protein solution (expressed in % wt. total protein relative to the total weight of the initial protein solution) advantageously can be kept at or below a certain threshold to facilitate the crystallisation of TP.

In some preferred embodiments of the invention, the ratio between the conductivity and the total amount of protein of the protein feeds is at most 0.3. Preferably, the ratio between the conductivity and the total amount of protein of the protein feeds is at most 0.25. Preferably, the ratio between the conductivity and the total amount of protein of the protein feeds is at most 0.20. More preferably, the ratio between the conductivity and the total amount of protein of the protein feeds is at most 0.18. Even more preferably, the ratio between the conductivity and the total amount of protein of the protein feeds is at most 0.12. Most preferably, the ratio between the conductivity and the total amount of protein of the protein feeds is at most 0.10.

It is for example preferred that the ratio between the conductivity and the total amount of protein of the protein feeds is approx. 0.07, or even lower.

The present inventors have furthermore found that the protein feed advantageously may be conditioned to obtain a UF permeate conductivity of at most 10 mS/cm. The UF permeate conductivity is a measure of the conductivity of the small molecule fraction of a liquid and is measured according to Example 9.10 of WO2018/115520. When the term “conductivity” is used herein as such it refers to the conductivity of the liquid in question. When the term “UF permeate conductivity” is used it refers to the conductivity of the small molecule fraction of a liquid and is measured according to Example 9.10 of WO2018/115520.

Preferably, the UF permeate conductivity of the protein feeds is at most 7 mS/cm. More preferably, the UF permeate conductivity of the protein feeds may be at most 5 mS/cm. Even more preferably, the UF permeate conductivity of the protein feeds may be at most 3 mS/cm.

Even lower UF permeate conductivities may be used and are particularly preferred if a high yield of TP should be obtained. Thus, preferably, the UF permeate conductivity of the protein feeds is at most 1.0 mS/cm. More preferably, the UF permeate conductivity of the protein feeds may be at most 0.4 mS/cm. Even more preferably, the UF permeate conductivity of the protein feeds may be at most 0.1 mS/cm. Most preferably, the UF permeate conductivity of the protein feeds may be at most 0.04 mS/cm.

Even lower UF permeate conductivities may reached, e.g. of MilliQ water is used as a diluent in during diafiltration (MilliQ water has a conductivity of approx. 0.06 μS/cm) Thus, the UF permeate conductivity of the protein feeds may be at most 0.01 mS/cm. Alternatively, the UF permeate conductivity of the protein feeds may be at most 0.001 mS/cm. Alternatively, the UF permeate conductivity of the protein feeds may be at most 0.0001 mS/cm.

In some preferred embodiments of the invention the preparation of the protein feeds involves a temperature reduction.

The temperature of the protein feeds may be at most 30 degrees C., preferably at most 20 degrees C., and even more preferably to at most 10 degrees C. The inventors have found that even lower temperatures provide higher degree of supersaturation obtain when the protein feeds are mixed, thus, the temperature of the protein feed may e.g. be reduced to at most 5 degrees C., preferably at most 2 degrees C., and even more preferably to at most 0 degrees C. The temperature may even be lower than 0 degrees C., however preferably the protein feeds should remain pumpable, e.g. in the form of an ice slurry.

In some preferred embodiments of the invention the protein feeds is an ice slurry before they are combined to prepared the initial protein solution.

The preparation of the protein feeds preferably involves one or more protein concentration steps such as ultrafiltration, nanofiltration, reverse osmosis, and/or evaporation.

Ultrafiltration is particularly preferred as it allows for selective concentration of protein. As mentioned above, ultrafiltration is preferably used both for diafiltration and concentration during the preparation of the protein feeds.

In some preferred embodiments of the invention, the concentration of TP of protein feeds is below the level where spontaneous crystallisation of TP occurs.

In some preferred embodiments of the invention the preparation of the protein feeds involves addition of one or more water activity reducing agent(s).

Useful, but non-limiting, examples of such water activity reducing agents are polysaccharides and/or poly-ethylene glycol (PEG).

In some preferred embodiments of the invention the preparation of the protein feeds involves ion exchange, by adding new ionic species, by dialysis or diafiltration.

In yet other preferred embodiments of the invention the preparation of the protein feeds involves reducing the conductivity, e.g. by diafiltration using a membrane that retains at least TP. Diafiltration by ultrafiltration is particularly preferred.

Any suitable protein source may be used to prepare the protein feeds. In some preferred embodiments of the present invention the protein source is produced by fermentation of one or more of mammal cells, recombinant plant cells, recombinant mammal cells, and/or recombinant microorganisms capable of producing TP.

Typically, the protein feeds are prepared by combining, and preferably mixing, two or more of the above-mentioned processes.

In some preferred embodiments of the invention the preparation of the protein feeds involves subjecting the protein source, to:
pH adjustment to the desired pH, preferably a pH in the range of pH_{MCY,TP}+0.5 to pH_{MCY,TP}+1.5 when preparing type A protein feeds and a pH in the range of pH_{MCY,TP}−1.5 to pH_{MCY,TP}−0.5 when preparing type B protein feeds, and
concentration by ultrafiltration, nanofiltration and/or reverse osmosis.

In other preferred embodiments of the invention the preparation of the protein feeds involves subjecting the protein source to:
pH adjustment to the desired pH, preferably a pH in the range of pH_{MCY,TP}+0.5 to pH_{MCY,TP}+1.5 when preparing type A protein feeds and a pH in the range of of pH_{MCY,TP}−1.5 to pH_{MCY,TP}−0.5 when preparing type B protein feeds,
concentration by ultrafiltration, nanofiltration and/or reverse osmosis,
ultrafiltration/diafiltration to provide a protein feed having a low conductivity, and
temperature adjustment to keep the temperature of the liquid streams during processing at at most 20 degrees C., more preferably at most 15 degrees C., even more preferably at most 10 degrees C., and most preferably at most 5 degrees C.

The present inventors have furthermore found that the TP yield of the present method may be improved by controlling the molar ratio between the sum of sodium+potassium vs. the sum of calcium and magnesium. A higher relative amount of calcium and magnesium surprisingly seems to increase the yield of TP and therefore increases the efficiency of the TP recovery of the present method.

In some preferred embodiments of the present invention the type A protein feed(s) would, if combined, have a molar ratio between Na+K and Ca+Mg of at most 4. More preferably, the type A protein feed(s) would, if combined, have a molar ratio between Na+K and Ca+Mg of at most 2. Even more preferably, the type A protein feed(s) would, if combined, have a molar ratio between Na+K and Ca+Mg of at most 1.5, and even more preferably at most 1.0. Most preferably, the type A protein feed(s) would, if combined, have a molar ratio between Na+K and Ca+Mg of at most 0.5, such as e.g. at most 0.2.

The molar ratio between Na+K and Ca+Mg it calculated as (m_{Na}+m_{K})/(m_{Ca}+m_{Mg}) wherein m_{Na} is the content of elemental Na in mol, m_{K} is the content of elemental K in mol, m_{Ca} is the content of elemental Ca in mol, and m_{Mg} is the content of elemental Mg in mol.

In some preferred embodiments of the present invention the type B protein feed(s) would, if combined, have a molar ratio between Na+K and Ca+Mg of at most 4. More preferably, the type B protein feed(s) would, if combined, have a molar ratio between Na+K and Ca+Mg of at most 2. Even more preferably, the type B protein feed(s) would, if combined, have a molar ratio between Na+K and Ca+Mg of at most 1.5, and even more preferably at most 1.0. Most preferably, the type A protein feed(s) would, if combined, have a molar ratio between Na+K and Ca+Mg of at most 0.5, such as e.g. at most 0.2.

In some embodiments of the invention the protein feeds have low content of denatured protein. Preferably, the protein feeds have a degree of protein denaturation of at most 20%, preferably at most 10%, more preferably at most 5%, and most preferably at most 2%.

In step a) the initial protein solution is prepared to have a pH at which the TP is capable of crystallizing under salting-in conditions, typically in the range of +/−0.7 pH-units of the pH_{MCY,TP}.

All pH values are measured using a pH glass electrode and are normalised to 25 degrees C.

More preferably the initial protein solution has a pH in the range of +/−0.5 pH-units of the pH_{MCY,TP}.

The inventors have observed that the maximum TP crystallisation yield is obtained at the pH_{MCY,TP} and that the pH of the initial protein solution advantageously can be selected outside the pH_{MCY,TP} but be moved closer to the optimum pH during the crystallisation, e.g. by addition of food acid or additional type B protein feed or type A protein feed. Thus, in some preferred embodiments of the present invention the pH of initial protein solution is in the range of pH_{MCY,TP}−0.7 to pH_{MCY,TP}−0.2. Alternatively but also preferred, the pH of initial protein solution may be in the range of pH_{MCY,TP}+0.2 to pH_{MCY,TP}+0.7.

In other preferred embodiments of the present invention the pH of initial protein solution is in the range of pH_{MCY,TP}−0.5 to pH_{MCY,TP}−0.2. Alternatively but also preferred, the pH of initial protein solution may be in the range of pH_{MCY,TP}+0.2 to pH_{MCY,TP}+0.5.

The inventors have found that it is advantageous to avoid spontaneous crystallization during the crystallization and that TP crystals formed by controlled crystallisation in the meta-stable zone have a more even size distribution and are easier to efficiently separate from the mother liquor that TP crystals resulting from spontaneous TP crystallisation.

Thus, in some preferred embodiments of the present invention the initial protein solution and preferably also the crystallizing protein solution are controlled to avoid spontaneous TP crystallisation. It is for example preferred that the initial protein solution is supersaturated with respect to TP but is in the meta-stable zone, meaning that spontaneous TP crystallisation cannot occur.

The inventors have found that a low conductivity of the initial protein solution leads to a higher yield of TP crystals. The minimum obtainable conductivity of the initial protein solution depends on the composition of the protein fraction and the lipid fraction (if any). Some protein species contribute more to the conductivity than other protein species. It is therefore preferable that the conductivity of the protein feed is brought near the level where protein and the counter ions of the protein are the main contributors to the conductivity. The reduction of conductivity often involves removal of at least some of the small, free ions that are present in liquid phase and not tightly bound to the proteins.

It is often preferred that the initial protein solution has a conductivity of at most 10 mS/cm. In some preferred embodiments of the invention, the initial protein solution has a conductivity of at most 5 mS/cm. Preferably, the initial protein solution has a conductivity of at most 4 mS/cm.

Lower conductivities are even more preferred and give rise to higher yields of TP crystals. Thus, the initial protein solution preferably has a conductivity of at most 3 mS/cm. In some preferred embodiments of the invention, the initial protein solution has a conductivity of at most 1 mS/cm. Preferably, the initial protein solution has a conductivity of at most 0.5 mS/cm.

The present inventors have seen indications that the ratio between the conductivity (expressed in mS/cm) and the total amount of protein of the initial protein solution (expressed in % wt. total protein relative to the total weight of the initial protein solution) advantageously can be kept at or below a certain threshold to facilitate the crystallisation of TP.

In some preferred embodiments of the invention, the ratio between the conductivity and the total amount of protein of the initial protein solution is at most 0.3. Preferably, the ratio between the conductivity and the total amount of protein of the initial protein solution is at most 0.25. Preferably, the ratio between the conductivity and the total amount of protein of the initial protein solution is at most 0.20. More preferably, the ratio between the conductivity and the total amount of protein of the initial protein solution is at most 0.18. Even more preferably, the ratio between the conductivity and the total amount of protein of the initial protein solution is at most 0.12. Most preferably, the ratio between the conductivity and the total amount of protein of the initial protein solution is at most 0.10.

It is for example preferred that the ratio between the conductivity and the total amount of protein of the initial protein solution is approx. 0.07, or even lower.

The present inventors have furthermore found that the initial protein solution advantegeously may have a UF permeate conductivity of at most 10 mS/cm. The UF permeate conductivity is a measure of the conductivity of the small molecule fraction of a liquid and is measured according to Example 9.10 of WO2018/115520. When the term “conductivity” is used herein as such it refers to the conductivity of the liquid in question. When the term “UF permeate conductivity” is used it refers to the conductivity of the small molecule fraction of a liquid and is measured according to Example 9.10 of WO2018/115520.

Preferably, the UF permeate conductivity of the initial protein solution is at most 7 mS/cm. More preferably, the UF permeate conductivity of the initial protein solution may be at most 5 mS/cm. Even more preferably, the UF permeate conductivity of the initial protein solution may be at most 3 mS/cm.

Even lower UF permeate conductivities may be used and are particularly preferred if a high yield of TP should be obtained. Thus, preferably, the UF permeate conductivity of the initial protein solution is at most 1.0 mS/cm. More preferably, the UF permeate conductivity of the initial protein solution may be at most 0.4 mS/cm. Even more preferably, the UF permeate conductivity of the initial protein solution may be at most 0.1 mS/cm. Most preferably, the UF permeate conductivity of the initial protein solution may be at most 0.04 mS/cm.

Even lower UF permeate conductivities may reached, e.g. of MilliQ water is used as a diluent in during diafiltration (MilliQ water has a conductivity of approx. 0.06 μS/cm) Thus, the UF permeate conductivity of the initial protein solution may be at most 0.01 mS/cm. Alternatively, the UF permeate conductivity of the initial protein solution may be at most 0.001 mS/cm. Alternatively, the UF permeate conductivity of the initial protein solution may be at most 0.0001 mS/cm.

The inventors have found that a low temperature increases the TP crystallisation yield. In some preferred embodiments of the present invention the temperature of the initial protein solution is at most 30 degrees C., more preferably at most 20 degrees C., and most preferably to at most 10 degrees C. Lower temperatures provide even higher degree of supersaturation, thus, the temperature of the initial protein solution is preferably at most 5 degrees C., more preferably at most 2 degrees C., and even more preferably at most 0 degrees C. The temperature may even be lower than 0 degrees C., however preferably the initial protein solution should remain pumpable, e.g. in the form of an ice slurry.

In some preferred embodiments of the invention, the initial protein solution is an ice slurry at the initialisation of TP crystallisation. Alternatively or additionally, crystallising initial protein solution may be converted into or maintained as an ice slurry during the TP crystallisation of step b).

It is particularly preferred that the initial protein solution is supersaturated with respect to the TP by salting-in and that TP therefore can be crystallised from the initial protein solution in salting-in mode.

In some embodiments of the invention the initial protein solution has low content of denatured protein, particularly if the TP product of the present invention should have degree of protein denaturation too. Preferably, the initial protein solution has a degree of protein denaturation of at most 20%, preferably at most 10%, more preferably at most 5%, and most preferably at most 2%.

Step b) of the method involves crystallising at least some of the TP of the supersaturated initial protein solution.

It is particularly preferred that the crystallisation of step b) takes place in salting-in mode, i.e. in a liquid that has a low ionic strength and conductivity. This is contrary to the salting-out mode wherein significant amounts of salts are added to a solution in order to provoke crystallisation.

The crystallisation of TP of step b) may e.g. involve one or more of the following:
Waiting for crystallisation to take place,
Addition of crystallisation seeds,
Increasing the degrees of supersaturation of TP even further, and/or
Mechanical stimulation.

In some preferred embodiments of the invention step b) involves adding crystallisation seeds to the initial protein solution. The inventors have found that addition of crystallisation seeds makes it possible to control when and where the TP crystallisation takes place to avoid sudden clogging of process equipment and unintentional stops during production. It is for example often desirable to avoid onset crystallisation while concentrating the protein feed.

In principle, any seed material which initiates the crystallisation of TP may be used. However, it is preferred that hydrated TP crystals or dried TP crystals are used for seeding to avoid adding additional impurities to the initial protein solution.

The crystallisation seeds may be on dry form or may form part of a suspension when added to the initial protein solution. Adding a suspension containing the crystallisation seeds, e.g. TP crystals, is presently preferred as it appears to provide a faster onset of crystallisation. It is preferred that such a suspension contain crystallisation seeds has a pH in the range of 5-6 and a conductivity of at most 10 mS/cm.

In some embodiments of the invention at least some of the crystallisation seeds are located on a solid phase which is brought in contact with the initial protein solution.

The crystallisation seeds preferably have a smaller particle size than the desired size of the TP crystals. The size of the crystallisation seeds may be modified by removing the largest seeds by sieving or other size fractionation processes. Particle size reduction, e.g. by means of grinding, may also be employed prior to the particle size fractionation.

In some embodiments of the invention at least 90% w/w of the crystallisation seeds have a particle size (measured by sieving analysis) in the range of 0.1-600 microns. For example, at least 90% w/w of the crystallisation seeds may have a particle size in the range of 1-400 microns. Preferably, at least 90% w/w of the crystallisation seeds may have a particle size in the range of 5-200 microns. More preferably, at least 90% w/w of the crystallisation seeds may have a particle size in the range of 5-100 microns.

The particle size and dosage of crystallisation seeds may be tailored to provide the optimal crystallisation of TP.

In some preferred embodiments of the invention at least one of the one or more type A or type B protein feed(s) already contains TP crystals in which case further seeding is not necessary, yet often preferred to ensure a sufficient amount of crystallisation seeds.

In some preferred embodiments of the invention step b) involves increasing the degree of supersaturation of TP even further, preferably to a degree of supersaturation where crystallisation of TP initiates immediately, i.e. in at most 20 minutes, and preferably in at most 5 minutes. This is also referred to as the nucleation zone wherein crystallites form spontaneously and start the crystallisation process.

The degree of supersaturation may e.g. be increased by one or more of the following:
increasing the protein concentration of the initial protein solution further
cooling the initial protein solution further
bringing the initial protein solution closer to the optimum pH for TP crystallisation
reducing the conductivity even further.

It is particularly preferred to increase the protein concentration of the initial protein solution further and/or to bring the initial protein solution closer to the optimum pH for TP crystallisation (pH_{MCY,TP}) by addition of additional type A or B protein feed.

In some preferred embodiments of the invention step b) involves waiting for the TP crystals to form. This may take several hours and is typically for an initial protein solution which is only slightly supersaturated with respect to TP and to which no crystallisation seeds have been added.

In some preferred embodiments of the invention the provision of the initial protein solution (step a) and the crystallisation of TP (step b) takes place as two separate steps. However, if the initial protein solution already contains at least some TP crystals step b) starts the moment the initial protein solution has been prepared.

In some preferred embodiments of the invention step b) involves additional adjustment of the crystallising protein solution preferably to raise the degree of supersaturation of TP, or at least maintain supersaturation. The additional adjustments preferably result in an increased yield of TP crystals or provide TP crystals of a better quality, e.g. in the form of a larger and more uniform crystal size.

Such additional adjustment may involve one or more of:
increasing the protein concentration of the crystallising protein solution even further cooling the crystallising protein solution to an even lower temperature
bringing the crystallising protein solution even closer to the optimum pH for TP crystallisation
reducing the conductivity of the crystallising protein solution even further.

In some preferred embodiments of the invention the crystallising protein solution is maintained in the meta-stable zone during step b) to avoid spontaneous formation of new crystallites.

It is particularly preferred to increase the protein concentration of the crystallizing protein solution further and/or to bring the crystallizing protein solution closer to the optimum pH for TP crystallisation (pH_{MCY,TP}) by addition of additional type A or B protein feed. The pH adjustment is preferably performed slowly to avoid spontaneous crystallisation or large local pH differences. Rates of pH adjustments in the range of 0.05-0.5 pH-units per hour have been found advantageous for controlled TP crystallization.

It is furthermore particularly preferred that the temperature of crystallizing protein solution is reduced during step b) to a temperature that is at least 2 degrees C. lower than the temperature of the initial protein solution, more preferably at least 4 degrees C. lower and most preferably at least 6 degrees C. lower than the temperature of the initial protein solution. It is however preferred that the crystallizing protein solution does not freeze completely.

The inventors have observed that the maximum TP crystallisation yield is obtained at pH_{MCY,TP} and that the pH of the initial protein solution advantageously can be selected outside the optimum but moved closer to the optimum pH during the crystallisation, e.g. by addition of food acid or more preferably by additional type B protein feed or type A protein feed.

Thus, in some preferred embodiments of the present invention the pH of initial protein solution is in the range of pH_{MCY,TP}−0.5 to pH_{MCY,TP}−0.2 or the range of pH_{MCY,TP}+0.2 to pH_{MCY,TP}+0.5. In such preferred embodiments of the present invention it is furthermore preferred to adjust the pH of the crystallizing protein solution to bring the pH of the crystallizing protein solution closer to the pH_{MCY,TP} than the pH of the initial protein solution, preferably by addition of additional type A protein feed or additional type B protein feed. It is particularly preferred that the crystallizing protein solution is adjusted to approx. pH_{MCY,TP} during the crystallisation. The additional type A protein feed or additional type B protein feed used for adjusting the crystallizing protein solution preferably has conductivity that is equal to or lower than the crystallizing protein solution. It is furthermore preferred to combine the above-mentioned pH adjustment during the crystallization with a reduction of the temperature of the crystallising protein solution so that the crystallizing protein solution is cooled to a temperature that is lower than the temperature of initial protein solution, e.g. simultaneously or sequentially.

In some preferred embodiments of the present invention:
the pH of initial protein solution of step a) is in the range of pH_{MCY,TP}−0.5 to pH_{MCY,TP}−0.2 or the range of pH_{MCY,TP}+0.2 to pH_{MCY,TP}+0.5 and the temperature of the initial protein solution is in the range of 5-20 degrees C.,
during step b) the pH of the crystallizing protein solution is adjusted to a pH in the range of pH_{MCY,TP}−0.1 to pH_{MCY,TP}+0.1, preferably by addition of additional type A protein feed if the pH of the initial protein solution is in the range of pH_{MCY,TP}−0.5 to pH_{MCY,TP}−0.2, or by addition of additional type B protein feed if the pH of the initial protein solution is in the range of pH_{MCY,TP}+0.2 to pH_{MCY,TP}+0.5, and
during step b) the temperature of crystallizing protein solution is reduced to a temperature that is at least 2 degrees C. lower than the temperature of the initial protein solution.

The terms “additional type A protein feed” and “additional type B protein feed” pertain to TP containing compositions that are added to the initial protein solution to provoke crystallisation or to the crystallizing protein solution to modify at least its pH and TP content. The features and preferences described in the context of a “type A protein feed” and a “type B protein feed” apply equally to an “additional type A protein feed” and an “additional type B protein feed”, respectively.

In some particularly preferred embodiments of the invention the method contains a step c) of separating at least some of the TP crystals from the remaining liquid of the TP crystal-containing solution. This is especially preferred when purification of TP is desired.

Step c) may for example comprise separating the TP crystals to a solids content of at least 30% w/w. Preferably, step c) comprises separating the TP crystals to a solids content of at least 40% w/w. Even more preferably step c) comprises separating the TP crystals to a solids content of at least 50% w/w.

The inventors have found that the high solids content is advantageous for the purification of TP as the aqueous portion that adhere to the separated TP crystals typically contains the impurities that should be avoided. Additionally, the high solids content reduces the energy consumption for converting the separated TP crystals to a dry product, such as e.g. a powder, and it increases the TP yield obtained from a drying unit with a given capacity.

In some preferred embodiments of the invention step c) comprises separating the TP crystals to a solids content of at least 60%. Preferably, step c) comprises separating the TP crystals to a solids content of at least 70%. Even more preferably step c) comprises separating the TP crystals to a solids content of at least 80%.

In some preferred embodiments of the invention the separation of step c) involves one or more of the following operations:
centrifugation,
decantation,
filtration,
sedimentation,
combinations of the above.

These unit operations are well-known to the person skilled in the art and are easily implemented. Separation by filtration may e.g. involve the use of vacuum filtration, dynamic crossflow filtration (DCF), a filtrate press or a filter centrifuge.

Different pore sizes for filtration may be employed based on the desired outcome. Preferably, the filter allows native proteins and small aggregates to pass but retains the TP crystals. The filter preferably has a nominal pore size of at least 0.1 micron. The filter may e.g. have a nominal pore size of at least 0.5 micron. Even more preferably, the filter may have a nominal pore size of at least 2 micron.

Filters having larger pore sizes can also be used and are in fact preferred if primarily the large crystals should be separated from a liquid containing TP crystals. In some embodiments of the invention the filter has a nominal pore size of at least 5 micron. Preferably, the filter has a nominal pore size of at least 20 micron. Even more preferably, the filter may have a pore size of at least 40 micron.

The filter may e.g. have a pore size in the range of 0.03-5000 micron, such as e.g. 0.1-5000 micron. Preferably, the filter may have a pore size in the range of 0.5-1000 micron. Even more preferably, the filter may have a pore size in the range of 5-800 micron, such as e.g. in the range of 10-500 micron or in the range of 50-500 microns.

In some preferred embodiments of the invention the filter has a pore size in the range of 0.03-100 micron. Preferably, the filter may have a pore size in the range of 0.1-50 micron. More preferably, the filter may have a pore size in the range of 4-40 micron. Even more preferably, the filter may have a pore size in the range of 5-30 micron such as in the range of 10-20 micron.

An advantage of using filters having a pore size larger than 1 micron is that bacteria and other microorganisms also are at least partly removed during separation and optionally also during washing and/or recrystallization. The present method therefore makes it possible to produce high purity TP with both a very low bacterial load yet avoiding heat-damage of the protein.

Another advantage of using filters having a pore size larger than 1 micron is that removal of water and subsequent drying becomes easier and less energy consuming.

The remaining liquid which is separated from the TP crystals may be recycled to one or more protein feed during preparation of the initial protein solution.

In some preferred embodiments of the invention, step c) employs a filter centrifuge. In other preferred embodiments of the invention, step c) employs a decanter centrifuge. Results (see Example 13 of WO2018/115520) have shown that use a filter centrifuge and/or a decanter centrifuge for separating TP crystals from the mother liquor provides more robust operation of the method than e.g. vacuum filtration.

Often it is preferred to dry a formed filter cake with a drying gas to reduce the moisture content of the filter cake and preferably to make it possible to peel the filter cake off the filter. The use of a drying gas may form part of the separation step or alternatively, the final drying step if the filter cake is converted directly to a dry TP product.

In some preferred embodiments of the invention, step c) employs a DCF unit.

In some preferred embodiments of the invention step c) is performed using a DCF unit equipped with a membrane capable of retaining TP crystals, the DCF permeate is recycle to form part of the initial protein solution or protein feed, and DCF retentate may be recovered or returned to the crystallization tank. Preferably, the DCF permeate is treated, e.g. by ultra-/diafiltration by to make it supersaturated with respect to TP prior to mixing with the initial protein solution or protein feed.

Advantageously, these embodiments do not require that the temperature of the liquid streams are raised above 15 degrees C. and are therefore less prone to microbial contamination than method variants that require higher temperatures. Another industrial advantage of the these embodiments is that the level of supersaturation is easily controlled and can be kept at a level where unwanted, spontaneous crystallization does not occur. The temperature of the liquid streams during these embodiments of the method is therefore preferably at most 15 degrees C., more preferred at most 12 degrees C., and even more preferred at most 10 degrees C., and most preferred at most 5 degrees C.

These embodiments are exemplified in Example 10 of WO2018/115520 and can equally be implemented with the TP. These embodiments may be implemented as a batch methods or a continuous method.

In some preferred embodiments of the invention the method comprises a step d) of washing TP crystals, e.g. the separated TP crystals of c). The washing may consist of a single wash or of multiple washing steps.

The washing of step d) preferably involves contacting the TP crystals with a washing liquid without completely dissolving the TP crystals and subsequently separating the remaining TP crystals from the washing liquid.

The washing liquid is preferably selected to avoid complete dissolution of the TP crystals and may e.g. comprise, or even consist essentially of, cold demineralised water, cold tap water, or cold reverse osmosis permeate.

The washing liquid may have a pH in the range of pH_{MCY,TP}−0.7 to pH_{MCY,TP}+0.7, preferably in the range of pH_{MCY,TP}−0.5 to pH_{MCY,TP}+0.5, and even more preferably in the range of pH_{MCY,TP}−0.4 to pH_{MCY,TP}+0.4, such as e.g. in the range of pH_{MCY,TP}−0.3 to pH_{MCY,TP}+0.3.

The washing liquid may have a conductivity of at most 0.1 mS/cm, preferably at most 0.02 mS/cm, and even more preferably at most 0.005 mS/cm.

Washing liquids having even lower conductivities may be used. For example, the washing liquid may have a conductivity of at most 1 microS/cm. Alternatively, the washing liquid may have a conductivity of at most 0.1 microS/cm, such as e.g. approx. 0.05 microS/cm.

A washing step is preferably performed at low temperature to limit the dissolution of crystallised TP. The temperature of the washing liquid is preferably at most 30 degrees C., more preferably at most 20 degrees C. and even more preferably at most 10 degrees C.

A washing step may e.g. be performed at at most 5 degrees C., more preferably at at most 2 degrees C. such as e.g. approx. 0 degrees C. Temperatures lower than 0 degrees C. may be used in so far that the washing liquid does not freeze at that temperature, e.g. due to the presence of one or more freezing point depressant(s).

In some embodiments of the invention the washing liquid contains TP, e.g. in an amount of at least 1% w/w, and preferably in an amount of at least 3% w/w, such as e.g. in an amount of 4% w/w.

The washing of step d) typically dissolves at most 80% w/w of the initial amount of TP crystals, preferably at most 50% w/w, and even more preferably at most 20% w/w of the initial amount of TP crystals. Preferably, the washing of step d) dissolves at most 15% w/w of the initial amount of TP crystals, more preferably at most 10% w/w, and even more preferably at most 5% w/w of the initial amount of TP crystals.

The weight ratio between the total amount of washing liquid and the initial amount of separated TP crystals is often at least 1, preferably at least 2 and more preferably at least 5. For example, the weight ratio between the amount of washing liquid and the initial amount of separated TP crystals may be at least 10. Alternatively, the weight ratio between the amount total of washing liquid and the initial amount of separated TP crystals may be at least 20, such as e.g. at least 50 or at least 100.

The term “total amount of washing liquid” pertains to the total amount of washing liquid used during the entire process.

In some preferred embodiments of the invention the one or more washing sequences take place in the same filter arrangement or in a similar filter arrangement as the TP crystal separation. A filter cake primarily containing TP crystals is added one or more sequences of washing liquid which is removed through the filter while the remaining part of the TP crystals stays in the filter cake.

In particularly preferred embodiments of the invention, the separation of step c) is performed using a filter that retains TP crystals. Subsequently, the filter cake is contacted with one or more quantities of washing liquid which moves through the filter cake and the filter. It is often preferred that each quantity of washing liquid is at most 10 times the volume of the filter cake, preferably at most 5 times the volume of the filter cake, more preferably at most 1 times the volume of the filter cake, even more preferably at most 0.5 times the volume of the filter cake, such as e.g. at most 0.2 times the volume of the filter cake. The volume of the filter cake includes both solids and fluids (liquids and gasses) of the filter cake. The filter cake is preferably washed this way at least 2 times, preferably at least 4 times and even more preferably at least 6 times.

The used washing liquid from step d) may e.g. be recycled to the protein feed or the initial protein solution where washed out TP may be isolated again.

The method may furthermore comprise a step e) which involves a recrystallization step. The recrystallization step may e.g. comprise:
dissolving the separated TP crystals in a recrystallization liquid,
adjusting the recrystallization liquid to obtain supersaturation with respect to TP,
crystallising TP in the supersaturated, adjusted recrystallization liquid, and
separating TP crystals from the remaining adjusted recrystallization liquid.

Alternatively and often more preferred, the separated TP crystals are converted into one or more type A protein feed(s) and one or more type B protein feed(s) which are combined to form a new supersaturated protein solution as in step a) and in which new protein solution TP is crystallised again.

Step e) may comprise either a single re-crystallisation sequence or multiple re-crystallisation sequences.

In some embodiments of the invention the TP crystals of step or c) or d) are recrystallized at least 2 times. For example, the TP crystals may be recrystallized at least 3 times, such as e.g. at least 4 times.

The washing and re-crystallization steps may be combined in any sequence and may be performed multiple times if required.

The separated TP crystals of step c) may e.g. be subjected to the process sequence:
One or more steps of washing (step d), followed by
One or more steps of re-crystallisation (step e).

Alternatively, the separated TP crystals of step c) may be subjected to the process sequence:
One or more steps of re-crystallisation (step e), followed by
One or more steps of washing (step d).

It is also possible to combine multiple steps of washing and re-crystallisation, e.g. in the sequence:
One or more steps of washing (step d),
One or more steps of re-crystallisation (step e),
One or more steps of washing (step d), and
One or more steps of re-crystallisation (step e).

Or e.g. in the sequence:
One or more steps of re-crystallisation (step e),
One or more steps of washing (step d),
One or more steps of re-crystallisation (step e).
One or more steps of washing (step d)

In some embodiments of the invention the method furthermore involves subjecting the separated TP to additional TP enrichments steps, e.g. based on chromatography or selective filtration. However, in other preferred embodiments of the invention the method does not contain additional TP enrichment steps after step b). By the term “additional TP enrichment step” is meant a process step which enriches TP relative to the total amount of protein, which step is not related to crystallisation of TP or handling of TP crystals. An example of such an additional TP enrichment step is ion exchange chromatography. Washing of TP crystals and/or recrystallization of TP is not considered “additional TP enrichment steps”.

In some particularly preferred embodiments of the invention the method involves a drying step f) wherein a TP-containing composition derived from steps b), c), d), or e) is converted to a dry product.

In the context of the present invention, the term “dry” means that the composition or product in question comprises at most 6% w/w water and preferably even less.

In the context of the present invention, the term “TP-containing composition” is used to describe the composition that is subjected to the drying of step f).

In the context of the present invention, a “TP-containing composition derived from step b), c), d), or e)” means a composition which comprises at least some of the TP from step b), c), d), or e). In some preferred embodiments of the invention the “TP-containing composition derived from step b), c), d), or e)” is directly obtained from step b), c), d), or e). However, in other preferred embodiments of the invention the “TP-containing composition derived from step b), c), d), or e)” is the result of further processing of the composition obtained directly from step b), c), d), or e).

It is often preferred that the TP-containing composition contains a significant amount of the TP present in the composition obtained directly from step b), c), d), or e). In some preferred embodiments of the invention the TP-containing composition derived from step b), c), d), or e) comprises at least 50% w/w of the TP obtained from step b), c), d), or e), preferably at least 70%, and even more preferably at least 80%.

Preferably, the TP-containing composition derived from step b), c), d), or e) comprises at least 85% w/w of the TP obtained from step b), c), d), or e). More preferably, the TP-containing composition derived from step b), c), d), or e) comprises at least 90% w/w of the TP obtained from step b), c), d), or e). Even more preferably, the TP-containing composition derived from step b), c), d), or e) comprises at least 95% w/w of the TP obtained from step b), c), d), or e). Most preferably, the TP-containing composition derived from step b), c), d), or e) comprises 100% w/w of the TP obtained from step b), c), d), or e).

In some preferred embodiments of the invention the drying step involves one or more of spray drying, freeze drying, spin-flash drying, rotary drying, and/or fluid bed drying.

In some particularly preferred embodiments of the invention the drying step involves a TP-containing composition in which the TP crystal has been dissolved and wherein the resulting powder does not contain TP crystals formed by step b) or by re-crystallisation prior to the drying step.

The TP crystals may e.g. be dissolved by:
increasing temperature,
increasing the conductivity, e.g. by addition of one or more salts
changing the pH, e.g. outside the range pH_{MCY,TP}−0.5 to pH_{MCY,TP}+0.5, and more preferably outside the range pH_{MCY,TP}−0.7 to pH_{MCY,TP}+0.7,
decreasing the concentration of TP, e.g. by dilution,
or a combination of the above.

Spray-drying is the presently preferred method of drying the TP-containing composition which does not contain TP crystals.

In other particularly preferred embodiments of the invention the drying step involves a TP-containing composition which still contains TP crystals and wherein the resulting powder contains TP crystals. These embodiments are preferred if the TP product should have a higher density than conventional, dried TP powder.

In some particularly preferred embodiments of the invention the drying step involves a TP-containing composition which still contains TP crystals and wherein the resulting powder contains TP crystals. These embodiments are preferred if the TP product should have a higher density than conventional, dried TP powder.

In some embodiments of the invention the TP-containing composition containing TP crystals has a temperature of at most 70 degrees C. when reaching the exit of the spray device (e.g. a nozzle or an atomizer), preferably at most 60 degrees C., more preferably at most 50 degrees C. In some preferred embodiments of the invention the TP-containing composition containing TP crystals has a temperature of at most 40 degrees C. when reaching the exit of the spray-device, preferably at most 30 degrees C., more preferably at most 20 degrees C., even more preferably at most 10 degrees C., and most preferably at most 5 degrees C.

The spray-device of the spray-dryer is the device, e.g. the nozzle or the atomizer, which converts the solution or suspension to be dried into droplets that enter the drying chamber of the spray-drier.

It is particularly preferred that the TP-containing composition containing TP crystals has a temperature in the range of 0-50 degrees C. when reaching the exit of the spray-device, preferably in the range of 2-40 degrees C., more preferably in the range of 4-35 degrees C., and most preferably in the range of 5-10 degrees C. when reaching the exit of the spray-device.

In some preferred embodiments of the invention, the TP-containing composition has a crystallinity of TP of at least 20% when reaching the exit of the spray-device, preferably at least 40%, more preferably at least 60%, even more preferably at least 80%, and a most preferably at least 90%, such as e.g. preferably 97-100%. TP-containing composition may either be a TP isolate, e.g. contain TP in an amount of more than 90% w/w relative to total protein or it may contain significant amounts of other proteins and therefore contain TP in an amount of at most 90% w/w relative to total protein.

The inlet temperature of gas of the spray-drier is preferably in the range of 140-220 degrees C., more preferably in the range of 160-200 degrees C., and even more preferably in the range of 170-190 degrees C., such as e.g. preferably approximately 180 degrees C. The exit temperature of the gas from the spray-drier is preferably in the range of 50-95 degrees C., more preferably in the range of 70-90 degrees C., and even more preferably in the range of 80-88 degrees C., such as e.g. preferably approximately 85 degrees C. As a rule of thumb, the solids that are subjected to spray-drying are said to be heated to a temperature which is 10-15 degrees C. less than the gas exit temperature.

In some preferred embodiments of the invention, the spray-drier is preferably in the range of 50-85 degrees C., more preferably in the range of 60-80 degrees C., and even more preferably in the range of 65-75 degrees C., such as e.g. preferably approximately 70 degrees C.

In some preferred embodiments of the invention the TP-containing composition to be dried is mixed with a dry TP isolate to raise the solids content to a level where the mixture can be dried by fluid bed drying. This is also referred to as back-mixing and allows for very cost efficient drying of the TP product. These embodiments are particularly preferred for TP-containing compositions that contain TP crystals.

An advantage of the present method is that the TP-containing composition to be dried may have a very high solids content prior to the drying step and therefore less water has to be removed and less energy is consumed in the drying operation.

In some preferred embodiments of the invention the TP-containing composition derived from step b), c), d), or e) has a solids content of at least 20% w/w. Preferably, the TP-containing composition derived from step b), c), d), or e) has a solids content of at least 30% w/w. More preferably, the TP-containing composition derived from step b), c), d), or e) has a solids content of at least 40% w/w. Even more preferably, the TP-containing composition derived from step b), c), d), or e) has a solids content of at least 50% w/w, such as e.g. at least 60% w/w.

In other preferred embodiments of the invention the TP-containing composition derived from step b), c), d), or e) has a solids content of in the range of 20-80% w/w. Preferably, the TP-containing composition derived from step b), c), d), or e) has a solids content in the range of 30-70% w/w. More preferably, the TP-containing composition derived from step b), c), d), or e) has a solids content in the range of 40-65% w/w. Even more preferably, the TP-containing composition derived from step b), c), d), or e) has a solids content in the range of 50-65% w/w, such as e.g. approx. 60% w/w.

The present inventors have found that the higher the crystallinity of the TP-containing composition, the less water is bound to the TP-containing composition, and the higher total solids content of the TP-containing composition can be achieved prior to the drying step.

Thus in some preferred embodiments of the invention, the TP-containing composition, has a crystallinity of TP of at least 10% w/w. Preferably, the TP of the TP-containing composition has a crystallinity of at least 20% w/w. More preferably the TP of the TP-containing composition has a crystallinity of at least 30% w/w. Even more preferably the TP of the TP-containing composition has a crystallinity of at least 40% w/w.

Even higher crystallinities are often preferred. Thus, in some preferred embodiments of the invention the TP of the TP-containing composition has a crystallinity of at least 50% w/w. Preferably, the TP of the TP-containing composition has a crystallinity of at least 60% w/w. More preferably, the TP of TP composition has a crystallinity of at least 70% w/w. Even more preferably, the TP of the TP-containing composition has a crystallinity of at least 80% w/w. Most preferred, the TP of the TP-containing composition has a crystallinity of at least 90% w/w, preferably at least 95% w/w, more preferably at least 97% w/w, and even more preferably at least 99% w/w.

The inventors have found that a reduced content of water tends to increase the crystallinity of TP of a composition. Thus, compositions having a high water:TP ratio (e.g. a suspension of 4% TP crystals in water) tend to have a lower crystallinity of TP than does compositions that have a lower water: TP ratio (e.g. a filter cake or moist, isolated crystals) at the same conditions.

The method of the present invention may be operated using mild temperatures that do not damage the TP.

In some preferred embodiments of the invention, the TP is not subjected to a temperature above 90 degrees C. during the method. Preferably, the TP is not subjected to a temperature above 80 degrees C. during the method. Even more preferred, the TP is not subjected to a temperature above 75 degrees C. during the method. It should be noted that even though spray-drying often employs temperatures in the excess of 150 degree C., the short exposure time and the concurrent evaporation of water means that the spray-dried proteins do not experience temperatures above 50-70 degrees C.

The inventors have seen indications that extended heating during the drying step reduces the amount of TP that is in crystal form. In some preferred embodiments of the invention the heat exposure during the drying step is kept sufficiently low to provide a degree of denaturation of TP of at most 10%, preferably at most 4%, more preferably at most 1%, even more preferably at most 0.4% and even more preferred at most 0.1%. Most preferably, the drying step does not result in detectable denaturation of TP at all.

The degree of denaturation caused by the drying step is calculated by determining the TP content (relative to total solids) in the TP-containing composition to be dried (c_{before step f}) in step f) and the TP content (relative to total solids) in the redissolved, dried composition and using the formula:

$$Degree of denaturation = ( ( {c}_{before step f} - {c}_{after step f} ) / {c}_{before step f} ) * 100 \%$$

An advantage of the present method is that it is much faster than comparable methods for TP crystallisation of the prior art. The duration from the initial adjustment of the protein feed to the completion of the separation of step c may be at most 10 hours, preferably at most 4 hours, more preferably at most 2 hours, and even more preferably at most 1 hour.

The TP product preferably contains TP in an amount of at least 30% w/w relative to the total amount of protein and an amount of total protein of at least 30% w/w relative to total solids.

In some preferred embodiments of the invention the TP of the TP product has a crystallinity of at least 10% w/w. Preferably, the TP of the TP product has a crystallinity of at least 20% w/w. More preferably the TP of the TP product has a crystallinity of at least 30% w/w. Even more preferably the TP of the TP product has a crystallinity of at least 40% w/w.

Even higher crystallinities are often preferred. Thus, in some preferred embodiments of the invention the TP of the TP product has a crystallinity of at least 50% w/w. Preferably, the TP of the TP product has a crystallinity of at least 60% w/w. More preferably, the TP of the TP product has a crystallinity of at least 70% w/w. Even more preferably, the TP of the TP product has a crystallinity of at least 80% w/w. Most preferred, the TP of the TP product has a crystallinity of at least 90% w/w, and preferably at least 95% w/w.

The crystallinity of TP in a liquid having pH in the range of pH_{MCY,TP}-0.5 to pH_{MCY,TP}+0.5 is measured as outlined in Example 9.7 of WO2018/115520 but measuring TP instead of BLG The crystallinity of TP in a powdered material is measured as outlined in Example 9.8 of WO2018/115520 but measuring TP instead of BLG. If the product is a dry product but not in the form a powder, it must be converted to a powder, e.g. by grinding or milling, before it is subjected to the method of Example 9.8 of WO2018/115520.

The TP product may e.g. comprise at most 90% w/w TP relative to the total amount of protein, and have a crystallinity of TP of at least 10%. For example, the TP product may comprise at most 80% w/w TP relative to the total amount of protein, and have a crystallinity of TP of at least 10%. The TP product may e.g. comprise 30-70% w/w TP relative to the total amount of protein, and have a crystallinity of TP of at least 10%.

In other preferred embodiments of the invention, the TP product comprises at most 90% w/w TP relative to the total amount of protein, and have a crystallinity of TP of at least 30%. Preferably, the TP product may comprise at most 80% w/w TP relative to the total amount of protein, and have a crystallinity of TP of at least 30%. Even more preferably, the TP product may comprise 30-70% w/w TP relative to the total amount of protein, and have a crystallinity of TP of at least 30%.

The present inventors have found that the present invention makes it possible to prepare an TP product having a very low content of phosphorus and other minerals, which is advantageous for patients suffering from kidney diseases or otherwise having a reduced kidney function.

The TP product is preferably a low phosphorus product.

In the context of the present invention the term “low phosphorus” pertains to a product, e.g. a liquid, a powder or another food product, that has a total content of phosphorus of at most 100 mg phosphorus per 100 g protein. Preferably, a low phosphorus product has a total content of at most 80 mg phosphorus per 100 g protein. More preferably, a low phosphorus product may have a total content of at most 50 mg phosphorus per 100 g protein. Even more preferably, a low phosphorus product may have a total content of phosphorus of at most 20 mg phosphorus per 100 g protein. Even more preferably, a low phosphorus product may have a total content of phosphorus of at most 5 mg phosphorus per 100 g protein. Low phosphorus products according to the present invention may be used as a food ingredient for the production of a food product for patients groups that have a reduced kidney function.

Thus, in some particularly preferred embodiments of the invention the TP product comprises at most 80 mg phosphorus per 100 g protein. Preferably, the TP product comprises at most 30 mg phosphorus per 100 g protein. More preferably, the TP product comprises at most 20 mg phosphorus per 100 g protein. Even more preferably, the TP product comprises at most 10 mg phosphorus per 100 g protein. Most preferably, the TP product comprises at most 5 mg phosphorus per 100 g protein.

The content of phosphorus relates to the total amount of elemental phosphorus of the composition in question and is determined according to Example 9.5 of WO2018/115520.

In other preferred embodiments of the invention the TP product is a low mineral composition.

In the context of the present invention the term “low mineral” pertains to a product, e.g. a liquid, a powder or another food product, that has at least one, preferably two, and even more preferably all, of the following:
an ash content of at most 1.2% w/w relative to total solids,
a total content of calcium and magnesium of at most 0.3% w/w relative to total solids,
a total content of sodium and potassium of at most 0.10% w/w relative to total solids,
a total content of phosphorus of at most 100 mg phosphorus per 100 g protein.

Preferably, a low mineral product has at least one, preferably two or more, and even more preferably all, of the following:
an ash content of at most 0.7% w/w relative to total solids,
a total content of calcium and magnesium of at most 0.2% w/w relative to total solids,
a total content of sodium and potassium of at most 0.08% w/w relative to total solids,
a total content of phosphorus of at most 80 mg phosphorus per 100 g protein.

Even more preferably, a low mineral product has at least one, preferably two or more, and even more preferably all, of the following:
an ash content of at most 0.5% w/w relative to total solids,
a total content of calcium and magnesium of at most 0.15% w/w relative to total solids,
a total content of sodium and potassium of at most 0.06% w/w relative to total solids,
a total content of phosphorus of at most 50 mg phosphorus per 100 g protein.

It is particularly preferred that a low mineral product has the following:
an ash content of at most 0.5% w/w relative to total solids,
a total content of calcium and magnesium of at most 0.15% w/w relative to total solids,
a total content of sodium and potassium of at most 0.06% w/w relative to total solids,
a total content of phosphorus of at most 50 mg phosphorus per 100 g protein.

In some preferred embodiments of the invention the TP product comprises a total amount of protein of at least 25% w/w relative to the total solids of the TP product. Preferably, the TP product comprises a total amount of protein of at least 50% w/w relative to the total solids of the TP product. More preferred, the TP product comprises a total amount of protein of at least 75% w/w relative to the total solids of the TP product. Even more preferred, the TP product comprises a total amount of protein of at least 90% w/w relative to the total solids of the TP product.

In some preferred embodiments of the invention the total amount of protein of the TP product is in the range of 25-100% w/w relative to total solids. Preferably, the total amount of protein of the TP product is in the range of 50-100% w/w. More preferred, the total amount of protein of the TP product is in range of 75-100% w/w relative to total solids. Even more preferred, the total amount of protein of the TP product is in the range of 90-100% w/w relative to total solids.

In some preferred embodiments of the invention the TP product comprises at least 75% w/w TP relative to the total amount of protein. Preferably, the TP product may comprise at least 90% w/w TP relative to the total amount of protein. More preferably, the TP product may comprise at least 95% w/w TP relative to the total amount of protein. Even more preferably, the TP product may comprise at least 97% w/w TP relative to the total amount of protein. Most preferably, the TP product comprises approx. 100% w/w TP relative to the total amount of protein.

In some preferred embodiments of the invention the TP product contains at most 10% w/w carbohydrate, preferably at most 5% w/w carbohydrate, more preferably at most 1% w/w carbohydrate, and even more preferably at most 0.1% w/w carbohydrate.

The TP product may also comprise lipid, e.g. in the form of triglyceride and/or other lipid types such as phospholipids.

In some embodiments of the invention the TP product comprises a total amount of lipid of at most 1% w/w relative to total solids. Preferably, the TP product comprises a total amount of lipid of at most 0.5% w/w relative to total solids. More preferably, the TP product comprises a total amount of lipid of at most 0.1% w/w relative to total solids. Even more preferably, the TP product comprises a total amount of lipid of at most 0.05% w/w relative to total solids. Most preferably, the TP product comprises a total amount of lipid of at most 0.01% w/w relative to total solids.

In some preferred embodiments of the invention the TP product is a dry product, and e.g. a powder. It is particularly preferred that the TP product is a spray-dried powder.

The present inventors have seen indications that TP products in powder form in which at least some of the TP is in crystal form when dried will have a higher bulk density than comparable TP product without TP crystals.

Thus, in some preferred embodiments of the invention the TP product in powder form has a bulk density of at least 0.40 g/mL. Preferably the TP product in powder form has a bulk density of at least 0.45 g/mL. More preferably the TP product in powder form has a bulk density of at least 0.50 g/mL. It is even more preferred that the TP product in powder form has a bulk density of at least 0.6 g/mL. The TP product in powder form may e.g. have a bulk density of at least 0.7 g/mL.

In some preferred embodiments of the invention the TP product in powder form has a bulk density of at least 0.45 g/mL and comprises at least 70% w/w protein relative to the total weight of the product. More preferably the TP product in powder form has a bulk density of at least 0.50 g/mL and comprises at least 70% w/w protein relative to the total weight of the product. It is even more preferred that the TP product in powder form has a bulk density of at least 0.6 g/ml and comprises at least 70% w/w protein relative to the total weight of the product. The TP product in powder form may e.g. have a bulk density of at least 0.7 g/mL and comprises at least 70% w/w protein relative to the total weight of the product.

In other preferred embodiments of the invention the TP product in powder form has a bulk density of at least 0.45 g/mL and comprises at least 80% w/w protein relative to the total weight of the product. More preferably the TP product in powder form has a bulk density of at least 0.50 g/mL and comprises at least 80% w/w protein relative to the total weight of the product. It is even more preferred that the TP product in powder form has a bulk density of at least 0.6 g/mL and comprises at least 80% w/w protein relative to the total weight of the product. The TP product in powder form may e.g. have a bulk density of at least 0.7 g/mL and comprises at least 80% w/w protein relative to the total weight of the product.

The TP product in powder form may e.g. have a bulk density in the range of 0.40-1.5 g/mL and comprises at least 80% w/w protein relative to the total weight of the product. Preferably, the powdered TP product has a bulk density in the range of 0.45-1.0 g/mL and comprises at least 80% w/w protein relative to the total weight of the product. More preferably the powdered TP product may have a bulk density in the range of 0.50-0.9 g/mL and comprises at least 80% w/w protein relative to the total weight of the product. It is even more preferred that the powdered TP product has a bulk density in the range of 0.6-0.9 g/mL and comprises at least 80% w/w protein relative to the total weight of the product. The powdered TP product may e.g. have a bulk density in the range of 0.6-0.8 g/mL and comprises at least 80% w/w protein relative to the total weight of the product.

The inventors have found that the high density powders of the invention advantageously allows for more cost-effective packaging and logistics of the powder as less packaging material is required per kg powder and more powder (mass) can be transported by a given container or truck.

The TP product in powder form may e.g. have a bulk density in the range of 0.40-1.5 g/mL. Preferably, the powdered TP product has a bulk density in the range of 0.45-1.0 g/mL. More preferably the powdered edible TP product may have a bulk density in the range of 0.50-0.9 g/mL. It is even more preferred that the powdered TP product has a bulk density in the range of 0.6-0.9 g/mL. The powdered TP product may e.g. have a bulk density in the range of 0.6-0.8 g/mL.

In other preferred embodiments of the invention the TP product in powder form has a bulk density in the range of 0.50-1.5 g/mL. Preferably, the powdered TP product has a bulk density in the range of 0.55-1.0 g/mL. More preferably the powdered TP product may have a bulk density in the range of 0.60-1.0 g/mL. It is even more preferred that the powdered TP product has a bulk density in the range of 0.65-1.0 g/mL. The powdered TP product may preferably have a bulk density in the range of 0.70-1.0 g/mL.

The TP product in powder form may e.g. have a bulk density in the range of 0.40-1.5 g/mL and comprises at least 70% w/w protein relative to the total weight of the product. Preferably, the powdered TP product has a bulk density in the range of 0.45-1.0 g/mL and comprises at least 70% w/w protein relative to the total weight of the product. More preferably the powdered TP product may have a bulk density in the range of 0.50-0.9 g/mL and comprises at least 70% w/w protein relative to the total weight of the product. It is even more preferred that the powdered TP product has a bulk density in the range of 0.6-0.9 g/mL and comprises at least 70% w/w protein relative to the total weight of the product. The powdered TP product may e.g. have a bulk density in the range of 0.6-0.8 g/mL and comprises at least 70% w/w protein relative to the total weight of the product.

The TP product in powder form may e.g. have a bulk density in the range of 0.40-1.5 g/mL and comprises at least 80% w/w protein relative to the total weight of the product. Preferably, the powdered TP product has a bulk density in the range of 0.45-1.0 g/mL and comprises at least 80% w/w protein relative to the total weight of the product. More preferably the powdered TP product may have a bulk density in the range of 0.50-0.9 g/mL and comprises at least 80% w/w protein relative to the total weight of the product. It is even more preferred that the powdered TP product has a bulk density in the range of 0.6-0.9 g/mL and comprises at least 80% w/w protein relative to the total weight of the product. The powdered TP product may e.g. have a bulk density in the range of 0.6-0.8 g/mL and comprises at least 80% w/w protein relative to the total weight of the product.

In other preferred embodiments of the invention the TP product in powder form has a bulk density in the range of 0.50-1.5 g/mL and comprises at least 70% w/w protein relative to the total weight of the product. Preferably, the powdered TP product has a bulk density in the range of 0.55-1.0 g/mL and comprises at least 70% w/w protein relative to the total weight of the product. More preferably the powdered TP product may have a bulk density in the range of 0.60-1.0 g/mL and comprises at least 70% w/w protein relative to the total weight of the product. It is even more preferred that the powdered TP product has a bulk density in the range of 0.65-1.0 g/mL and comprises at least 70% w/w protein relative to the total weight of the product. The powdered TP product may preferably have a bulk density in the range of 0.70-1.0 g/mL and comprises at least 70% w/w protein relative to the total weight of the product.

In other preferred embodiments of the invention the TP product in powder form has a bulk density in the range of 0.50-1.5 g/mL and comprises at least 80% w/w protein relative to the total weight of the product. Preferably, the powdered TP product has a bulk density in the range of 0.55-1.0 g/mL and comprises at least 80% w/w protein relative to the total weight of the product. More preferably the powdered TP product may have a bulk density in the range of 0.60-1.0 g/mL and comprises at least 80% w/w protein relative to the total weight of the product. It is even more preferred that the powdered TP product has a bulk density in the range of 0.65-1.0 g/mL and comprises at least 80% w/w protein relative to the total weight of the product. The powdered TP product may preferably have a bulk density in the range of 0.70-1.0 g/mL and comprises at least 80% w/w protein relative to the total weight of the product.

The bulk density of a powder is measured according to Example 9.3 of WO2018/115520.

In some preferred embodiments of the invention the TP product is a liquid composition. A liquid TP product preferably comprises at least 20% w/w water, more preferably at least 30% w/w water, even more preferably at least 40% w/w.

The liquid TP product may e.g. comprises in the range of 20-90% w/w water, more preferably in the range of 30-80% w/w water, even more preferably at least 40% w/w.

The present inventors have seen indications that TP products according to the present invention have surprisingly low degree of protein denaturation, even if spray-drying has been used to prepare an TP powder product.

Thus, in some preferred embodiments of the invention the TP product has a degree of protein denaturation of at most 10%. Preferably, the TP product has a degree of protein denaturation of at most 5%. More preferably, the TP product has a degree of protein denaturation of at most 2%. Even more preferably, the TP product has a degree of protein denaturation of at most 1%. Even more preferably, the TP product has a degree of protein denaturation of at most 0.5%.

In some preferred embodiments of the invention, the TP product is a dry powder, and preferably a spray-dried powder, and has a degree of protein denaturation of at most 2%, and preferably at most 1.5%. More preferably, the dry TP product, e.g. in the form of a spray-dried powder, has a degree of protein denaturation of at most 1.0%. Even more preferably, the dry TP product, e.g. in the form of a spray-dried powder, has a degree of protein denaturation of at most 0.8%. Even more preferably, the dry TP product, e.g. in the form of a spray-dried powder, has a degree of protein denaturation of at most 0.5%.

In some preferred embodiments of the invention, the TP product comprises:
At most 6% w/w water
At least 80% total protein relative to total solids
At least 95% TP relative to total protein, and said TP product:
Is a dry powder, and
Has a bulk density of at least 0.50 g/mL, and preferably at least 0.60 g/mL.

In other preferred embodiments of the invention, the TP product comprises:
At most 6% w/w water
At least 80% total protein relative to total solids
At least 95% TP relative to total protein, and said TP product:
Is a dry powder,
Has a bulk density of at least 0.50 g/mL, and preferably at least 0.60 g/mL, and
Has a crystallinity of TP of at least 20% and preferably at least 40%.

In further preferred embodiments of the invention, the TP product comprises:
At most 6% w/w water
At least 80% total protein relative to total solids
At least 95% TP relative to total protein, and said TP product:
Is a dry powder,
Has a bulk density of at least 0.50 g/mL, and preferably at least 0.60 g/mL, and
Has a degree of protein denaturation of at most 2%, and preferably at most 1.0%.

In further preferred embodiments of the invention, the TP product comprises:
At most 6% w/w water
At least 80% total protein relative to total solids,
At least 95% TP relative to total protein,
at most 80 mg phosphorus per 100 g protein. said TP product:
Is a dry powder.

In yet preferred embodiments of the invention, the TP product comprises:
At most 6% w/w water
At least 90% total protein relative to total solids,
At least 97% TP relative to total protein,
at most 50 mg phosphorus per 100 g protein. said TP product:
Is a dry powder.

In other preferred embodiments of the invention, the TP product comprises:
At most 6% w/w water
At least 80% total protein relative to total solids, and preferably at least 90% total protein relative to total solids,
30-90% TP relative to total protein, said TP product:
Is a dry powder, and
Has a crystallinity of TP of at least 20% and preferably at least 40%.

In some preferred embodiments of the invention, the TP product comprises:
20-80% w/w water, and preferably 20-60% w/w water,
At least 80% total protein relative to total solids, and preferably at least 90% total protein
At least 95% TP relative to total protein,
at most 80 mg phosphorus per 100 g protein. said TP product:
has a crystallinity of TP of at least 20%, preferably at least 40, and
optionally, has a degree of protein denaturation of at most 2%, and preferably at most 1.0%.

Liquid TP products according to these embodiments are particularly useful for preparing TP products in dried form, and are particularly suitable for spray-drying and preparation of a high density TP protein powder having the normal concentration profile of other protein species but containing at least some of the TP in the form of dried TP crystals.

In other preferred embodiments of the invention, the TP product comprises:
20-80% w/w water, and preferably 20-60% w/w water,
At least 80% total protein relative to total solids, and preferably at least 90% total protein relative to total solids,
30-79% TP relative to total protein, said TP product:
Has a crystallinity of TP of at least 20% and preferably at least 40%.

products according to these embodiments are particularly useful for preparing TP products in dried form, and are particularly suitable for spray-drying and preparation of a high density TP protein powder having the normal concentration profile of other protein species but containing at least some of the TP in the form of dried TP crystals.

The present method is preferably performed at a temperature in the range of 1-65 degrees C., preferably 2-50 degrees C., more preferably in the range of 3-20 degrees C., even more preferably in the range of 4-15 degrees C.

Another aspect of the invention pertains to the TP product obtainable by the present method.

The present invention has been described above with reference to specific embodiments. However, other embodiments than the above described are equally possible within the scope of the invention. The different features and steps of various embodiments and aspects of the invention may be combined in other ways than those described herein unless it is stated otherwise.

### EXAMPLES

### Example 1: Crystallization of Beta-Lactoglobulin (BLG) Based on Mixing of Two WPI Streams

### INTRODUCTION

During the initial trials of large-scale implementation of the method of WO2018/115520, frequent events of spontaneous BLG crystallization were observed in the ultrafiltration unit used to prepare the whey protein solution. The events of spontaneous BLG crystallization led to interruptions of the production and to time-consuming cleaning of the process units affected by the crystallization.

The new approach in relation to BLG (described in PCT/EP2022/053010):

The inventors then had the idea that instead of producing the supersaturated protein solution of WO2018/115520 from a single feed that could become supersaturated with respect to BLG under unfavorable conditions during preparation (e.g. at the occurrence of high, local protein concentration and/or sudden cooling), it was smarter to produce the supersaturated whey protein solution from two (or more) feeds, each having a pH that makes it clearly non-supersaturated with respect to BLG (or only supersaturated to a limited degree), and then mixing to two (or more) feeds to get the right pH for supersaturation of BLG, i.e. a pH close to pH 5.5.

This new approach makes it possible to prepare protein feeds with a very high content of protein without the risk of uncontrolled BLG crystallisation. Additionally, this approach makes it possible to prepare the feeds, and subsequently the initial protein solution, at temperatures significantly below the growth optimum of microorganisms, which is advantageous as less heat-treatment is required to reduce the microbial content of the end products to an acceptable level. The inventors have furthermore found that the pH adjustments made by the present type A and/or type B protein feeds contributes only with a very limited change in the conductivity of the initial protein solution as opposed to making the same pH change with traditional food acids or food bases. This is beneficial as a reduced conductivity of the initial protein solution typically gives rise to a higher BLG crystallisation yield.

The new approach also applies to other protein species (the present invention):

The inventors have appreciated that the new approach disclosed in PCT/EP2022/053010 also applies to other protein species capable of crystallizing under salting-in conditions than BLG, in so far the pH ranges are adapted as described herein. The feasibility of the new approach is demonstrated in this Example using BLG as a model protein capable of crystallizing under salting-in conditions but can be applied to other protein species capable of crystallizing under salting-in conditions.

In the present example, the protein feeds were based on BLG-containing, whey protein isolates and the first protein feed (Batch One, a Type A protein feed) had a pH of 6.2 and the second protein feed (Batch Two, a Type B protein feed) had a pH of 4.9. Despite a high protein concentration of BLG, none of the protein feeds were supersaturated. However, by simply mixing the two feeds in appropriate amounts (to obtain a pH close to pH 5.5, which the inventors have found to be the pH that provides the maximum crystallisation yield of BLG, pH_{MCY,BLG}) initial protein solutions supersaturated with respect to BLG were obtained from which BLG could be crystallized.

### Process:

The whey raw material for this example was a lactose depleted UF retentate derived from sweet whey from a standard cheese production process, the whey had been fat reduced via a Synder FR membrane prior to use. From this raw material two batches was produced and conditioned by an ultrafiltration setup using a Alfa Laval GR82PE membrane with a 30 mill spacer and a feed pressure of 1.5-3.0 bar. The product was demineralized with polished water as diafiltration medium and the feed had a concentration of 18 percent TS (Total Solids)+5 percent. The diafiltration continued at least until the drop in conductivity in the retentate was below 0.02 mS/cm over a 20 min period where the TS in the retentate was stable (+0.5 percent TS). The retentate was then concentrated to around 22 percent TS the feed composition of Batch One and Two can be seen in Table 1.1 and 1.2.

The first batch (Batch One) was pH adjusted to pH 6.1 with a food grade hydrochloric acid (HCl) prior to UF treatment at 10 to 12 degrees C. The other batch (Batch Two) was pH adjusted to pH 4.9 with a food grade hydrochloric acid (HCl) prior to UF treatment at 10 to 12 degrees C.

TABLE 1.1
Data describing on Batch One
Batch One
Physical aspects:

| | |
|---|---|
| pH | 6.2 |
| TS (%) | 21.2 |
| Conductivity(mS/cm) | 2.52 |

Protein composition %
of total protein (%)

| | |
|---|---|
| ALA | 14 |
| BLG | 63 |
| other | 23 |

minerals a.o (%)
relative to TS

| | |
|---|---|
| Ca | 0.461 |
| K | 0.486 |
| Mg | 0.079 |
| Na | 0.151 |
| P | 0.217 |
| protein concentration | 90 |

ALA = alpha-lactalbumin.

TABLE 1.2
Data describing on Batch Two
Batch Two
Physical aspects:

| | |
|---|---|
| PH | 4.9 |
| TS (%) | 21.2 |
| Conductivity(mS/cm) | 0.71 |

Protein composition % of total protein (%)

| | |
|---|---|
| ALA | 14 |
| BLG | 60 |
| other | 26 |

minerals a.o (%) relative to TS

| | |
|---|---|
| Ca | 0.257 |
| K | 0.064 |
| Mg | 0.039 |
| Na | BDL* |
| P | 0.228 |
| protein concentration | 94 |

*BDL = Below Detection Limit

After UF treatment the temperature of the two batches was adjusted to 10 degrees C. before portions of the batches were mixed together in five different ratios to produce the protein solutions in which the crystallization was conducted see Table 1.3 for details. Each mixture was seeded at 10 degrees C. with 0.1% w/w seed material and cooled to 5 degrees C. after seeding and left over night with agitation.

In order to check whether the Batch One and Batch Two were already supersaturated, samples of each of the two batches were taken prior to the above-mentioned mixing and were also seeded and stored at 10 degrees C. overnight. Visual inspection by microscopy the following morning showed that no crystals were present after incubation and it can therefore be concluded that Batch One and Batch Two were not supersaturated.

The seed material was produced by mixing a small portion of Batch One and Two together to reach a final pH of 5.5 and subsequently add a small amount of dried BLG crystal material. The seed solution was then placed on ice for one hour with stirring.

Samples of the protein solutions were taken prior to seeding and after crystallization and centrifuge at 3000 g for 5 minute, samples of the supernatant were analyzed by RP-HPLC as describe in Example 1 of WO2018/115520 and the crystallization yield was calculated by the following formula:

$$Yield = 1 - ( \frac{( 1 - \% {BLG}_{before crystallization} )}{( 1 - \% {BLG}_{after crystallization} )} ) \times \frac{\% {BLG}_{after crystallization}}{\% {BLG}_{before crystallization}}$$

Where % BLG_{before crystallization} is the BLG concentration measured by HPLC in the sample prior to seeding ad crystallization and % BLG_{after crystallization} is the BLG concentration measured by HPLC in the supernatant of the sample after crystallization.

### Results:

As can be seen from the table below it is possible to condition whey protein feeds in an pH area where they have a low degree of supersaturation (or even no supersaturation) with respect to BLG thereby lowering the risk of spontaneous crystallization in the in the UF plant and increasing the maximum protein concentration that can be used without spontaneous BLG crystallization. The two feeds were subsequently mixed to provide an initial protein solution with a high degree of BLG supersaturation.

TABLE 1.3
Protein solution data.

| Solution: | 1 | 2 | 3 | 4 | 5 |
|---|---|---|---|---|---|
| pH | 5.2 | 5.3 | 5.5 | 5.7 | 5.8 |
| Conductivety | 1.43 | 1.58 | 1.81 | 2.18 | 2.32 |
| (mS/cm) | | | | | |
| Yield (%) | 35 | 62 | 68 | 54 | 28 |
| Mix ratio (Batch | 0.62 | | | 5.46 | |
| One:Batch Two) | | | | | |

The inventors appreciate that results of Example 1 demonstrate that any protein capable of crystallising under salting-in conditions can be crystallised according to the approach of Example 1 if the pH ranges of the feeds are modified relative to the pH_{MCY,TP} of protein as described herein.

### Example 2: Crystallization Based on Mixing of Two WPI Streams, Using a Commercial Available WPI

The whey material for this example was commercial available Lacprodan DI-9213 and a second self-produced pH 6.2 WPI.

The pH 6.2 WPI was made from a lactose depleted UF retentate derived from sweet whey from a standard cheese production process, the whey had been fat reduced via a Synder FR membrane prior to use. The raw material was pH adjusted to pH 6.2 with a food grade hydrochloric acid prior to UF treatment at 10 to 12 degrees C. From this raw material a batche was produced and conditioned by an ultrafiltration setup using a Alfa Laval GR82PE membrane with a 30 mill spacer and a feed pressure of 1.5-3.0 bar. The product was demineralized with polished water as diafiltration medium and the feed had a concentration of 18 percent TS±5 percent. The diafiltration continued at least until the drop in conductivity in the retentate was below 0.02 mS/cm over a 20 min period where the TS in the retentate was stable (±0.5 percent TS). The retentate was then concentrated to around 22 percent TS the feed composition of Batch One and Two can be seen in Tables 2.1 and 2.2.

The Batch Two was made from Lacprodan DI-9213 in powder form.

The powder was conditioned by rehydrating it to a TS of 16.3 and leaving it with stirring at 10 degrees C. until all powder was dissolved.

TABLE 2.1
Characteristics of Batch One
Batch one
Physical aspects:

| | |
|---|---|
| PH | 6.1 |
| TS (%) | 28.2 |
| Conductivity(mS/cm) | 2.73 |

Protein composition % of total protein (%)

| | |
|---|---|
| ALA | 9 |
| BLG | 62 |
| other | 29 |

minerals a.o (%) relative to TS

| | |
|---|---|
| Ca | 0.492 |
| K | 0.533 |
| Mg | 0.082 |
| Na | 0.123 |
| P | 0.205 |
| protein concentration | 89 |

TABLE 2.2
Data describing Batch Two
Batch Two
Physical aspects:

| | |
|---|---|
| pH | 3.4 |
| TS (%) | 16.3 |
| Conductivity(mS/cm) | 1.58 |

Protein composition % of total protein (%)

| | |
|---|---|
| ALA | 10 |
| BLG | 70 |
| other | 20 |

minerals a.o (%) relative to TS

| | |
|---|---|
| Ca | 0.019 |
| K | 0.003 |
| Mg | 0.003 |
| Na | 0.003 |
| P | 1.425 |
| protein concentration | 87.3 |

After conditioning the two batches was treated (with the exception that only two solutions were made) and analyzed as in Example 1.

The characteristics of the mixtures can be seen in Table 2.3.

### Results:

As can be seen from the table below it is possible to condition whey protein feeds in a pH area where they have a no supersaturation with regards to BLG thereby lowering the risk of spontaneous crystallization and increasing the level protein concentration possible without risking crystallization in the UF plant, and then subsequently mix the two together in order to obtain a high degree of supersaturation. It can be seen that the higher conductivity impacts the yield as expected.

TABLE 2.3
Protein solution data.

| solution: | 1 | 2 |
|---|---|---|
| PH | 5.4 | 5.5 |
| Conductivety | 2.57 | 2.58 |
| (mS/cm) | | |
| Yield (%) | 55 | 56 |

The inventors appreciate that results of Example 2 demonstrate that any protein capable of crystallising under salting-in conditions can be crystallised according to the approach of Example 2 if the pH ranges of the feeds are modified relative to the pH_{MCY,TP} of protein as described herein.

### Example 3: Crystallization Based on Mixing of Two WPI Streams, pH 4.0 and 6.1

The whey raw material for this example was a lactose depleted UF retentate derived from sweet whey from a standard cheese production process, the whey had been fat reduced via a Synder FR membrane prior to use. From this raw material two batches was produced and conditioned by an ultrafiltration setup using a Alfa Laval GR82PE membrane with a 30 mill spacer and a feed pressure of 1.5-3.0 bar. The product was demineralized with polished water as diafiltration medium and the feed had a concentration of 18 percent TS ±5 percent. The diafiltration continued at least untill the drop in conductivity in the retentate was below 0.02 mS/cm over a 20 min period where the TS in the retentate was stable (±0.5 percent TS). The retentate was then concentrated to around 22 percent TS the feed composition of Batch One and Two can be seen in Tables 3.1 and 3.2.

One batch (batch one) was pH adjusted to pH 6.1 with a food grade hydrochloric acid (HCl) prior to UF treatment at 10 to 12 degrees C. The other batch (batch two) was pH adjusted to pH 4.0 with a food grade hydrochloric acid (HCl) prior to UF treatment at 10 to 12 degrees C.

TABLE 3.1
Data describing on Batch One
Batch One
Physical aspects:

| | |
|---|---|
| pH | 6.1 |
| TS (%) | 21.2 |
| Conductivity(mS/cm) | 2.52 |

Protein composition % of total protein (%)

| | |
|---|---|
| ALA | 14 |
| BLG | 63 |
| other | 23 |

minerals a.o (%) relative to TS

| | |
|---|---|
| Ca | 0.461 |
| K | 0.486 |
| Mg | 0.079 |
| Na | 0.151 |
| P | 0.217 |
| protein concentration | 90 |

TABLE 3.2
data describing on Batch Two
Batch Two
Physical aspects:

| | |
|---|---|
| pH | 4.0 |
| TS (%) | 22.2 |
| Conductivity(mS/cm) | 1.49 |

Protein composition % of total protein (%)

| | |
|---|---|
| ALA | 12 |
| BLG | 67 |
| other | 21 |

minerals a.o (%) relative to TS

| | |
|---|---|
| Ca | BDL* |
| K | BDL* |
| Mg | BDL* |
| Na | BDL* |
| P | 0.202 |
| protein concentration | 89 |

*BDL = Below Detection Limit

After conditioning the two batches was treated (with the exception that only four solutions were made) and analyzed as in Example 1 of WO2018/115520.

The characteristics of the mixtures can be seen in Table 3.3.

### Results:

As can be seen from the table below it is possible to condition whey protein feeds in an area where they have a low degree of supersaturation with regards to BLG thereby lowering the risk of spontaneous crystallization and increasing the level protein concentration possible without risking crystallization in the UF plant, and then subsequently mix the two together in order to obtain a high degree of supersaturation.

TABLE 3.3
Protein solution data.

| solution: | 1 | 2 | 3 | 4 |
|---|---|---|---|---|
| pH | 5.25 | 5.40 | 5.55 | 5.70 |
| Conductivity | 2.3 | 2.34 | 2.42 | 2.46 |
| (mS/cm) | | | | |
| Yield (%) | 33 | 56 | 56 | 48 |

The inventors appreciate that results of Example 3 demonstrate that any protein capable of crystallising under salting-in conditions can be crystallised according to the approach of Example 3 if the pH ranges of the feeds are modified relative to the pH_{MCY,TP} of protein as described herein.

### Example 4: Crystal Size Distribution

The whey raw material for this example was a lactose depleted UF retentate derived from sweet whey from a standard cheese production process, the whey had been fat reduced via a Synder FR membrane prior to use. From this raw material two batches for each crystallization was produced and conditioned by an ultrafiltration setup using a Alfa Laval GR82PE membrane with a 30 mill spacer and a feed pressure of 1.5-3.0 bar. The product was demineralized with polished water as diafiltration medium and the feed had a concentration of 18 percent TS ±5 percent. The diafiltration continued at least untill the drop in conductivity in the retentate was below 0.02 mS/cm over a 20 min period where the TS in the retentate was stable (±0.5 percent TS). The retentate was then concentrated to around 22 percent TS the feed composition of batch one and two can be seen in the tables below

One batch (Batch One) was pH adjusted to pH around 6.1 with a food grade hydrochloric acid (HCl) prior to UF treatment at 10 to 12 degrees C. The other batch (Batch Two) was pH adjusted to pH 4.8 with a food grade hydrochloric acid (HCl) prior to UF treatment at 10 to 12 degrees C.

The crystallizations were conducted in a 300L crystallisation tank and 0.1% w/w seed material was used. The seed material was produces as in Example 1.

1^{st} Crystallization

TABLE 4.1
Data describing on Batch One
Batch One
Physical aspects:

| | |
|---|---|
| pH | 6.1 |
| TS (%) | 23.2 |
| Conductivity(mS/cm) | 2.72 |

Protein composition % of total protein (%)

| | |
|---|---|
| ALA | 14 |
| BLG | 63 |
| other | 23 |

minerals a.o (%) relative to TS

| | |
|---|---|
| Ca | 0.492 |
| K | 0.533 |
| Mg | 0.082 |
| Na | 0.123 |
| P | 0.205 |
| protein concentration | 89 |

TABLE 4.2
Data describing on Batch Two
Batch Two
Physical aspects:

| | |
|---|---|
| pH | 4.8 |
| TS (%) | 23.3 |
| Conductivity(mS/cm) | 0.72 |

Protein composition % of total protein (%)

| | |
|---|---|
| ALA | 10 |
| BLG | 62 |
| other | 28 |

minerals a.o (%) relative to TS

| | |
|---|---|
| Ca | 0.248 |
| K | 0.082 |
| Mg | 0.041 |
| Na | BDL* |
| P | 0.206 |
| protein concentration | 90 |

*BDL = Below Detection Limit

After conditioning the two batches were mixed together to obtain a final pH of 5.50, the mixture was seeded and rapidly cooled to 5 degrees C. and left over Night to crystallize. Samples of the slurry was analysed as described in example 1 to calculate the yield. The particle size distribution of the crystal slurry was analysed by Malvern particles sizes characterization.

### 2^{nd} Crystallization

TABLE 4.3
Data describing on Batch One
Batch One
Physical aspects:

| | |
|---|---|
| pH | 6.2 |
| TS (%) | 23.2 |
| Conductivity(mS/cm) | 2.70 |

Protein composition % of total protein (%)

| | |
|---|---|
| ALA | 10 |
| BLG | 62 |
| other | 28 |

minerals a.o (%) relative to TS

| | |
|---|---|
| Ca | 0.452 |
| K | 0.492 |
| Mg | 0.072 |
| Na | 0.150 |
| P | 0.205 |
| protein concentration | 88 |

TABLE 4.4
Data describing on Batch Two
Batch Two
Physical aspects:

| | |
|---|---|
| pH | 4.8 |
| TS (%) | 23.3 |
| Conductivity(mS/cm) | 0.82 |

Protein composition % of total protein (%)

| | |
|---|---|
| ALA | 10 |
| BLG | 62 |
| other | 28 |

minerals a.o (%) relative to TS

| | |
|---|---|
| Ca | 0.214 |
| K | 0.067 |
| Mg | 0.030 |
| Na | BDL* |
| P | 0.196 |
| protein concentration | 90 |

*BDL = Below Detection Limit

After conditioning the two batches was mixed together to obtain a final pH of 5.45, the mixture was seeded and rapidly cooled to 5 degrees C. and left over Night to crystallize. Samples of the slurry was analysed as described in example 1 to calculate the yield. The particle size distribution of the crystal slurry was analysed by Malvern particles size characterization.

### 3^{rd} Crystallization:

TABLE 4.5
Data describing on Batch One
Batch one
Physical aspects:

| | |
|---|---|
| pH | 6.1 |
| TS (%) | 22.9 |
| Conductivity(mS/cm) | 2.66 |

Protein composition % of total protein (%)

| | |
|---|---|
| ALA | 9 |
| BLG | 66 |
| other | 25 |

minerals a.o (%) relative to TS

| | |
|---|---|
| Ca | 0.405 |
| K | 0.485 |
| Mg | 0.073 |
| Na | 0.149 |
| P | 0.177 |
| protein concentration | 87 |

TABLE 4.6
Data describing on Batch Two
Batch Two
Physical aspects:

| | |
|---|---|
| pH | 4.8 |
| TS (%) | 23.3 |
| Conductivity(mS/cm) | 0.82 |

Protein composition % of total protein (%)

| | |
|---|---|
| ALA | 10 |
| BLG | 69 |
| other | 21 |

minerals a.o (%) relative to TS

| | |
|---|---|
| Ca | 0.192 |
| K | 0.061 |
| Mg | 0.029 |
| Na | BDL* |
| P | 0.176 |
| protein concentration | 89 |

*BDL = Below Detection Limit

After conditioning the two batches was mixed together to obtain a pH of 5.80, the mixture was seeded and incubated for 1 hour before it was adjusted, by further addition of Batch Two, over 2 hours to a final pH of 5.45 and then rapidly cooled to 5 degrees C. and left over Night to crystallize. Samples of the slurry was analysed as described in Example 1 to calculate the yield. The particle size distribution of the crystal slurry was analysed by Malvern particles size characterization.

### Results:

In the Table 4.7 the results of the crystallizations are shown, and as can be seen the crystal size distributions were similar even though different raw materials were used and the crystallization processes differed.

TABLE 4.7
Overview of crystallization results

| | BLG | | | |
|---|---|---|---|---|
| Crystallization | Yield (%) | Dx(10) | Dx(50) | Dx(90) |
| 1 | 73 | 80 | 167 | 263 |
| 2 | 75 | 58 | 153 | 253 |
| 3 | 71 | 57 | 127 | 248 |

The inventors appreciate that results of Example 4 demonstrate that any protein capable of crystallising under salting-in conditions can be crystallised according to the approach of Example 4 if the pH ranges of the feeds are modified relative to the pH_{MCY,TP} of protein as described herein.

### Example 5

Examples 1-4 of U.S. Pat. No. 5,719,048 can be modified according to the present invention as described below.

### Example 1 of U.S. Pat. No. 5,719,048

The diafiltrate of the Example 1 of U.S. Pat. No. 5,719,048 is divided into two batches and pH-adjusted, and further diafiltrered to prepare:
a Type A feed having a pH of 5.1, a conductivity of 3 mS/cm and containing 13% w/w of the lipase, and
a Type B-feed having a pH of 3.9, a conductivity of 3 mS/cm and containing 13% w/w of the lipase.

The initial protein solution of step a) of the present invention is prepared by mixing the type A feed with an amount of the type-B feed sufficient to provide a pH of 4.5 in the mixture.

### Example 2 of U.S. Pat. No. 5,719,048

The diafiltrate of the Example 2 of U.S. Pat. No. 5,719,048 is divided into two batches and pH-adjusted, and further diafiltrered to prepare:
a Type A feed having a pH of 4.9, a conductivity of 3 mS/cm and containing 11% w/w of the lipase, and
a Type B-feed having a pH of 3.9, a conductivity of 3 mS/cm and containing 11% w/w of the lipase.

The initial protein solution of step a) of the present invention is prepared by mixing the type A feed with an amount of the type B feed sufficient to provide a pH of 4.3 in the mixture.

### Example 3 of U.S. Pat. No. 5,719,048

The diafiltrate of the Example 3 of U.S. Pat. No. 5,719,048 is divided into two batches and pH-adjusted, and further diafiltrered to prepare:
a Type A feed having a pH of 5.6, a conductivity of 3 mS/cm and containing 10% w/w of the lipase, and
a Type B-feed having a pH of 4.4, a conductivity of 3 mS/cm and containing 10% w/w of the lipase.

The initial protein solution of step a) of the present invention is prepared by mixing the type A feed with an amount of the type B feed sufficient to provide a pH of 5.0 in the mixture.

### Example 4 of U.S. Pat. No. 5,719,048

The diafiltrate of the Example 4 of U.S. Pat. No. 5,719,048 is divided into two batches and pH-adjusted, and further diafiltrered to prepare:
a Type A feed having a pH of 4.9, a conductivity of 3 mS/cm and containing 10% w/w of the lipase, and
a Type B-feed having a pH of 3.9, a conductivity of 3 mS/cm and containing 10% w/w of the lipase.

The initial protein solution of step a) of the present invention is prepared by mixing the type A feed with an amount of the type B feed sufficient to provide a pH of 4.3 in the mixture.

1. A method of preparing a product, preferably an edible or pharmaceutically acceptable product, comprising a target protein (TP) capable of crystallizing under salting-in conditions, the method comprising the steps of:
a) preparing an initial protein solution comprising the TP, said initial protein solution is supersaturated with respect to the TP and has a pH at which the TP is capable of crystalizing under salting-in conditions,
b) crystallising the TP in the supersaturated, initial protein solution in salting-in mode thereby obtaining a TP crystal-containing solution, and
c) preferably, separating TP crystals from the remaining liquid of the TP crystal-containing solution,
wherein step a) involves preparing the initial protein solution by combining one or more type A protein feed(s) with one or more type B protein feed(s),
and wherein:
a “type A protein feed” is defined as a protein feed which comprises the TP and has a pH that is at least 0.1 pH unit higher than the pH_{MCY,TP}, and
a “type B protein feed” is defined as a protein feed which comprises the TP and has a pH that is at least 0.1 pH unit lower than the pH_{MCY,TP},
and preferably with the proviso that the TP is not a beta-lactoglobulin.

2. The method according to claim 1 wherein:
in relation to type A protein feed:
if only a single type A protein feed is used it has, or
if two or more type A protein feeds are used they would, if mixed, have
a pH of at least pH_{MCY,TP}+0.1, more preferably at least pH_{MCY,TP}+0.2, even more preferably at least pH_{MCY,TP}+0.3, and most preferably at least pH_{MCY,TP}+0.4, and
in relation to type B protein feed:
if only a single type B protein feed is used it has, or
if two or more type B protein feeds are used they would, if mixed, have
a pH of at most pH_{MCY,TP}−0.1, more preferably at most pH_{MCY,TP}−0.2, even more preferably at most pH_{MCY,TP}−0.3, and most preferably at most pH_{MCY,TP}−0.4.

3. The method according to any one of the preceding claims, wherein:
in relation to type A protein feed:
if only a single type A protein feed is used it has, or
if two or more type A protein feeds are used they would, if mixed, have
a pH of at least pH_{MCY,TP}+0.5, more preferably at least pH_{MCY,TP}+0.6, even more preferably at least pH_{MCY,TP}+0.7, and most preferably at least pH_{MCY,TP}+0.8, and
in relation to type B protein feed:
if only a single type B protein feed is used it has, or
if two or more type B protein feeds are used they would, if mixed, have
a pH of at most pH_{MCY,TP}−0.5, more preferably at most pH_{MCY,TP}−0.6, even more preferably at most pH_{MCY,TP}−0.7, and most preferably at most pH_{MCY,TP}−0.8.

4. The method according to any one of the preceding claims, wherein:
in relation to type A protein feed:
if only a single type A protein feed is used it has, or
if two or more type A protein feeds are used they would, if mixed, have
a pH of at least pH_{MCY,TP}+0.9, more preferably at least pH_{MCY,TP}+1.0, even more preferably at least pH_{MCY,TP}+1.2, and most preferably at least pH_{MCY,TP}+1.5, and
in relation to type B protein feed:
if only a single type B protein feed is used it has, or
if two or more type B protein feeds are used they would, if mixed, have
a pH of at most pH_{MCY,TP}−0.9, more preferably at most pH_{MCY,TP}−1.0, even more preferably at most pH_{MCY,TP}−1.2, and most preferably at most pH_{MCY,TP}−1.5.

5. The method according to any one of the preceding claims, wherein in relation to type A protein feed:
if only a single type A protein feed is used it has, or
if two or more type A protein feeds are used they would, if mixed, have a pH of in the range of pH_{MCY,TP}+0.1 to pH_{MCY,TP}+4.5, more preferably pH_{MCY,TP}+0.3 to pH_{MCY,TP}+2.5, even more preferably pH_{MCY,TP}+0.5 to pH_{MCY,TP}+1.5 and most preferably pH_{MCY,TP}+0.6 to pH_{MCY,TP}+1.0.

6. The method according to any one of the preceding claims, wherein in relation to type A protein feed:
if only a single type A protein feed is used it has, or
if two or more type A protein feeds are used they would, if mixed, have a pH of in the range of pH_{MCY,TP}+0.5 to pH_{MCY,TP}+2.5, more preferably pH_{MCY,TP}+0.6 to pH_{MCY,TP}+2.0, even more preferably pH_{MCY,TP}+0.7 to pH_{MCY,TP}+1.5 and most preferably pH_{MCY,TP}+0.7 to pH_{MCY,TP}+1.0.

7. The method according to any one of the preceding claims, wherein in relation to type A protein feed:
if only a single type A protein feed is used it has, or
if two or more type A protein feeds are used they would, if mixed, have a pH of in the range of pH_{MCY,TP}+0.7 to pH_{MCY,TP}+3.0, more preferably pH_{MCY,TP}+0.8 to pH_{MCY,TP}+2.5, even more preferably pH_{MCY,TP}+0.9 to pH_{MCY,TP}+2.0 and most preferably pH_{MCY,TP}+1.0 to pH_{MCY,TP}+1.5.

8. The method according to any one of the preceding claims, wherein in relation to type B protein feed:
if only a single type B protein feed is used it has, or
if two or more type B protein feeds are used they would, if mixed, have a pH of in the range of pH_{MCY,TP}−3.5 to pH_{MCY,TP}−0.1, more preferably pH_{MCY,TP}−2.5 to pH_{MCY,TP}−0.3, even more preferably pH_{MCY,TP}−1.5 to pH_{MCY,TP}−0.5, and most preferably pH_{MCY,TP}−1.0 to pH_{MCY,TP}−0.6.

9. The method according to any one of the preceding claims, wherein in relation to type B protein feed:
if only a single type B protein feed is used it has, or
if two or more type B protein feeds are used they would, if mixed, have a pH of in the range of pH_{MCY,TP}−1.5 to pH_{MCY,TP}−0.5, more preferably pH_{MCY,TP}−1.4 to pH_{MCY,TP}−0.6, even more preferably pH_{MCY,TP}−1.2 to pH_{MCY,TP}−0.7, and most preferably pH_{MCY,TP}−1.0 to pH_{MCY,TP}−0.7.

10. The method according to any one of the preceding claims, wherein in relation to type B protein feed:
if only a single type B protein feed is used it has, or
if two or more type B protein feeds are used they would, if mixed, have a pH of in the range of pH_{MCY,TP}−2.0 to pH_{MCY,TP}−0.7, more preferably pH_{MCY,TP}−1.8 to pH_{MCY,TP}−0.8, even more preferably pH_{MCY,TP}−1.6 to pH_{MCY,TP}−0.9, and most preferably pH_{MCY,TP}−1.5 to pH_{MCY,TP}−1.0.

11. The method according to any one of the preceding claims,
wherein at least one of the one or more type A protein feeds does not contain TP crystals, more preferably none of the one or more type A protein feeds contain TP crystals, and/or wherein at least one of the one or more type B protein feeds do not contain TP crystals, more preferably none of the one or more type B protein feeds contain TP crystals.

12. The method according to any one of the preceding claims, wherein at least one of the one or more type A protein feeds is not supersaturated with respect to TP, and more preferably none of the one or more type A protein feeds are supersaturated with respect to TP and/or wherein at least one of the one or more type B protein feeds is not supersaturated with respect to TP, and more preferably none of the one or more type are supersaturated with respect to TP.

13. The method according to any one of the preceding claims, wherein type A and type B protein feed(s) that do not contain TP crystals contribute with at least 1% w/w of the TP content of the initial protein solution, more preferably at least 10% w/w, even more preferably at least 20% w/w, and most preferably at least 40% w/w.

14. The method according to any one of the preceding claims, wherein type A and type B protein feed(s) that do not contain TP crystals contribute with at least 50% w/w of the TP content of the initial protein solution, more preferably at least 70% w/w, even more preferably at least 80% w/w, and most preferably at least 90% w/w.

15. The method according to any one of the preceding claims, wherein in relation to type A protein feed:
if only a single type A protein feed is used it has, or
if two or more type A protein feeds are used they would, if mixed, have a temperature of at most 60 degrees C., more preferably at most 50 degrees C., even more preferably at most 40 degrees C. and most preferably at most 30 degrees C.

16. The method according to any one of the preceding claims, wherein in relation to type B protein feed:
if only a single type B protein feed is used it has, or
if two or more type B protein feeds are used they would, if mixed, have a temperature of at most 60 degrees C., more preferably at most 50 degrees C., even more preferably at most 40 degrees C. and most preferably at most 30 degrees C.

17. The method according to any one of the preceding claims, wherein in relation to type A protein feed:
if only a single type A protein feed is used it comprises, or
if two or more type A protein feeds are used they would, if mixed, comprise at least 1% w/w TP relative to the total weight of type A protein feed, more preferably at least 3% w/w, even more preferably at least 5% w/w, and most preferably at least 7% w/w TP relative to the total weight of type A protein feed.

18. The method according to any one of the preceding claims, wherein in relation to type B protein feed:
if only a single type B protein feed is used it comprises, or
if two or more type B protein feeds are used they would, if mixed, comprise at least 1% w/w TP relative to the total weight of type B protein feed, more preferably at least 3% w/w, even more preferably at least 5% w/w, and most preferably at least 7% w/w TP relative to the total weight of type B protein feed.

19. The method according to any one of the preceding claims,
wherein the initial protein solution comprises a total amount of type A protein feed in the range of 1-99% w/w relative to the weight of the initial protein solution, more preferably 10-80% w/w, even more preferably 20-70% w/w, and most preferably 30-60% w/w

20. The method according to any one of the preceding claims,
wherein the initial protein solution comprises a total amount of type B protein feed(s) in the range of 1-99% w/w relative to the weight of the initial protein solution, more preferably 10-80% w/w, even more preferably 20-70% w/w, and most preferably 30-60% w/w.

21. The method according to any one of the preceding claims, wherein the initial protein solution comprises:
a total amount of type A protein feed(s) having a pH of pH_{MCY,TP}+0.3 to pH_{MCY,TP}+2.5 in the range of 5-90% w/w relative to the weight of the initial protein solution, more preferably 10-80% w/w, even more preferably 20-70% w/w, and most preferably 30-60% w/w, and
a total amount of type B protein feed(s) having a pH of pH_{MCY,TP}−2.5 to pH_{MCY,TP}−0.3 in the range of 5-90% w/w relative to the weight of the initial protein solution, more preferably 10-80% w/w, even more preferably 20-70% w/w, and most preferably 30-60% w/w.

22. The method according to any one of the preceding claims, wherein the initial protein solution comprises:
a total amount of type A protein feed(s) having a pH of pH_{MCY,TP}+0.5 to pH_{MCY,TP}+2.5 in the range of 5-90% w/w relative to the weight of the initial protein solution, more preferably 10-80% w/w, even more preferably 20-70% w/w, and most preferably 30-60% w/w, and
a total amount of type B protein feed(s) having a pH of pH_{MCY,TP}−2.5 to pH_{MCY,TP}−0.5 in the range of 5-90% w/w relative to the weight of the initial protein solution, more preferably 10-80% w/w, even more preferably 20-70% w/w, and most preferably 30-60% w/w.

23. The method according to any one of the preceding claims, wherein the initial protein solution comprises:
a total amount of type A protein feed(s) having a pH of pH_{MCY,TP}+0.6 to pH_{MCY,TP}+1.0 in the range of 5-90% w/w relative to the weight of the initial protein solution, more preferably 10-80% w/w, even more preferably 20-70% w/w, and most preferably 30-60% w/w, and
a total amount of type B protein feed(s) having a pH of pH_{MCY,TP}−1.0 to pH_{MCY,TP}−0.6 in the range of 5-90% w/w relative to the weight of the initial protein solution, more preferably 10-80% w/w, even more preferably 20-70% w/w, and most preferably 30-60% w/w.

24. The method according to any one of the preceding claims, wherein the initial protein solution comprises:
a total amount of type A protein feed(s) having a pH of pH_{MCY,TP}+0.8 to pH_{MCY,TP}+1.5 in the range of 5-90% w/w relative to the weight of the initial protein solution, more preferably 10-80% w/w, even more preferably 20-70% w/w, and most preferably 30-60% w/w, and
a total amount of type B protein feed(s) having a pH of pH_{MCY,TP}−1.5 to pH_{MCY,TP}−0.8 in the range of 5-90% w/w relative to the weight of the initial protein solution, more preferably 10-80% w/w, even more preferably 20-70% w/w, and most preferably 30-60% w/w.

25. The method according to any one of the preceding claims, wherein type A protein feed(s) and type B protein feed(s) contribute with at least 50% w/w of the protein content of the initial protein solution, more preferably at least 70% w/w, even more preferably at least 80% w/w, and most preferably at least 90% w/w.

26. The method according to any one of the preceding claims, wherein type A protein feed(s) and type B protein feed(s) contribute with at least 92% w/w of the protein content of the initial protein solution, more preferably at least 94% w/w, even more preferably at least 96% w/w, and most preferably at least 99% w/w.

27. The method according to any one of the preceding claims, wherein type A protein feed(s) and type B protein feed(s) contribute with at least 50% w/w of the solids content of the initial protein solution, more preferably at least 70% w/w, even more preferably at least 80% w/w, and most preferably at least 90% w/w.

28. The method according to any one of the preceding claims, wherein type A protein feed(s) and type B protein feed(s) contribute with at least 92% w/w of the solids content of the initial protein solution, more preferably at least 94% w/w, even more preferably at least 96% w/w, and most preferably at least 99% w/w.

29. The method according to any one of the preceding claims, wherein type A protein feed(s) and type B protein feed(s) contribute with at least 50% w/w of the weight of the initial protein solution, more preferably at least 70% w/w, even more preferably at least 80% w/w, and most preferably at least 90% w/w.

30. The method according to any one of the preceding claims, wherein type A protein feed(s) and type B protein feed(s) contribute with at least 92% w/w of the weight of the initial protein solution, more preferably at least 94% w/w, even more preferably at least 96% w/w, and most preferably at least 99% w/w.

31. The method according to any one of the preceding claims, wherein the degree of supersaturation of TP of the initial protein solution is higher than both the degree of supersaturation of:
the type A protein feed if only a single type A protein feed is used, or
the type A protein feeds, if combined, if two or more type A protein feeds are used, and of:
the type B protein feed if only a single type B protein feed is used, or
the type B protein feeds, if combined, if two or more type B protein feeds are used.

32. The method according to any one of the preceding claims, wherein the initial protein solution has a pH in the range of +/−0.7 pH-units of the pH providing the maximum crystallisation yield of the TP (pH_{MCY,TP}), and most preferably in the range of +/−0.5 pH-units of the pH_{MCY,TP}.

33. The method according to any one of the preceding claims wherein the TP is a globular protein, and preferably a globulin or a globin.

34. The method according to any one of the preceding claims wherein the TP is selected from the group consisting of an enzyme, an immunoglobulin, a hormone.

35. The method according to any one of the preceding claims wherein the TP is an enzyme, preferably an oxidoreductase (EC 1), a transferase (EC 2), a hydrolase (EC 3), a lyase (EC 4), or an isomerase (EC 5).

36. The method according to any one of the preceding claims wherein the TP is an oxidoreductase enzyme (EC 1), preferably selected from one or more of the following a Laccase (EC 1.10.3.2), a Catalase (EC 1.11.1.6), a Glucose oxidase (EC 1.1.3.4), a Ligninase (EC 1.11.1.14) and a Peroxidase (EC 1.11.1).

37. The method according to any one of the preceding claims wherein the TP is a hydrolase enzyme (EC 3).

38. The method according to any one of the preceding claims wherein the TP is an esterase enzyme (EC 3.1), preferably selected from a Phytase (EC 3.1.3), a lipase (EC Number 3.1.1.3), phosphatase (EC 3.1.3), a phytase, and/or a nuclease (EC 3.1.11-3.1.31).

39. The method according to any one of the preceding claims wherein the TP is a glycosylase enzyme (EC 3.2), preferably selected from an alpha-amylase (EC 3.2.1.1), an Amyloglucosidase (EC 3.2.1.3 or EC 3.2.1.33), a Cellulase (EC 3.2.1.4), a Hemicellulase (EC 3.2.1.4), a Xylanase (EC 3.2.1.8), a Muramidase (EC 3.2.1.17), a Pullulanase (EC 3.2.1.41), a Pectinase (EC 3.2.1.67), a beta-galactosidase (EC 3.2.1.23), a lactase (EC 3.2.1.108), a Glucoamylase (EC 3.2.1.20 or EC 3.2.1.3), and a Dextranase (EC 3.2.1.11).

40. The method according to any one of the preceding claims wherein the TP is a peptidase or protease enzyme (EC 3.4), e.g. selected from a chymotrypsin (EC 3.4.21.1), a trypsin, a pepsin, a chymosin (EC 3.4.23.4), or an alkaline protease.

41. The method according to any one of the preceding claims wherein the TP is a lyase enzyme (EC 4), e.g. a pectate lyase or an alpha-acetolactate decarboxylase.

42. The method according to any one of the preceding claims wherein the TP is an isomerase enzyme (EC 5), e.g. a glucose isomerase.

43. The method according to any one of the preceding claims wherein the TP is a hormone, preferably human insulin (uniprot ID: P01308), a human insulin analogue, a human insulin agonist, human GLP-1, a human GLP-1 analogue, or a human GLP-1 agonist.

44. The method according to any one of the preceding claims wherein the TP is an immunoglobulin, and preferably a monoclonal immunoglobulin.

45. The method according to any one of the preceding claims wherein the TP is an immunoglobulin G (IgG), and preferably a monoclonal IgG.

46. The method according to any one of the preceding claims wherein the TP is a bispecific IgG.

47. The method according to any one of the preceding claims wherein the TP is an interferon, an interferon analogue, or an interferon agonist.

48. The method according to any of claims 30-32 wherein the TP is a therapeutic protein selected from the group consisting of Muromonab-CD3, Abciximab, Rituximab, Palivizumab, Infliximab, Trastuzumab, Alemtuzumab, Adalimumab, Ibritumomab tiuxetan, Omalizumab, Cetuximab, Bevacizumab, Natalizumab, Panitumumab, Ranibizumab, Eculizumab, Certolizumab pegol, Ustekinumab, Canakinumab, Golimumab, Ofatumumab, Tocilizumab, Denosumab, Belimumab, Ipilimumab, Brentuximab vedotin, Pertuzumab, Trastuzumab emtansine, Raxibacumab, Obinutuzumab, Siltuximab, Ramucirumab, Vedolizumab, Blinatumomab, Nivolumab, Pembrolizumab, Idarucizumab, Necitumumab, Dinutuximab, Secukinumab, Mepolizumab, Alirocumab, Evolocumab, Daratumumab, Elotuzumab, Ixekizumab, Reslizumab, Olaratumab, Bezlotoxumab, Atezolizumab, Obiltoxaximab, Inotuzumab ozogamicin, Brodalumab, Guselkumab, Dupilumab, Sarilumab, Avelumab, Ocrelizumab, Emicizumab, Benralizumab, Gemtuzumab ozogamicin, Durvalumab, Burosumab, Lanadelumab, Mogamulizumab, Erenumab, Galcanezumab, Tildrakizumab, Cemiplimab, Emapalumab, Fremanezumab, Ibalizumab, Moxetumomab pasudodox, Ravulizumab, Caplacizumab, Romosozumab, Risankizumab, Polatuzumab vedotin, Brolucizumab, and Crizanlizumab.

49. The method according to any one of the preceding claims wherein the TP is prepared by purification from a natural source, by fermentation of a recombinant organism, and/or by subsequent protein rearrangement.

50. The method according to any one of the preceding claims wherein the TP is a therapeutic protein.

51. A TP product obtainable by the method according to one or more of claims 1-50.
